(19) United States
(12) Patent Application Publication
(10) Pub. No.: US 2026/0053369 A1
(43) Pub. Date: Feb. 26, 2026

(54) HANDHELD PROBE AND SYSTEM FOR IMAGING HUMAN TISSUE

(71) Applicant: Optican Systems Inc., West Vancouver (CA)

(72) Inventors: Farid Golnaraghi, West Vancouver (CA); Ramani Ramasehan, Vancouver (CA); Shadi Momtahen, Vancouver (CA); Maryam Momtahen, Vancouver (CA); Ryley McWilliams, Vancouver (CA); Mohammad Pouriayevali, Vancouver (CA); Avner Bachar, Vancouver (CA)

(21) Appl. No.: 19/308,126

(22) Filed: Aug. 22, 2025

Related U.S. Application Data
(60) Provisional application No. 63/687,242, filed on Aug. 26, 2024.

(51) Int. Cl. A61B 5/00; H04N 25/20

(52) U.S. Cl. CPC A61B 5/0075; A61B 5/4312; A61B 5/7203; A61B 5/7267; A61B 2560/0431; A61B 2562/0233; A61B 2562/043; A61B 2576/02; H04N 25/20

(57) ABSTRACT

A portable optical spectroscopy system may provide noninvasive imaging of human tissue. The system may comprise a multi-wavelength near-infrared emitter source including at least one multi-chip light source configured to sequentially emit light at multiple wavelengths. A local controller may be configured to control a light emission sequence at two or more wavelengths. A sensor may be aligned in reflectance geometry with the emitter sources and configured to detect diffusely reflected near infrared light from biological tissue. A remote processor may be configured to reconstruct two-dimensional images of tissue optical properties in real time. Sequential NIR illumination from at least one source may be aligned with the sensor detector. The multi-wavelength near-infrared emitter source may comprise wavelengths selected from 670 nm, 810 nm, and 950 nm corresponding to absorption characteristics of deoxyhemoglobin, oxyhemoglobin, water, and fat. The system may generate cross-sectional images displaying spatial distribution of tissue constituents.

### PRIORITY CLAIM

This application claims the benefit of U.S. Provisional Application No. 63/687,242 entitled “IMPROVED HANDHELD PROBE AND SYSTEM FOR IMAGING HUMAN TISSUE” filed Aug. 26, 2024, which is incorporated by reference in its entirety herein for all purposes.

### BACKGROUND

According to the Canadian and American Cancer Societies'reports, breast cancer may be the most common cancer in women. Breast cancer may be ranked as the second cause of death in North America. Breast cancer may affect approximately one in eight North American women during their lifetime. Diagnosis and treatment may involve several steps, from the initial assessment to post-treatment follow-up. Each step may be taken seriously, particularly when a lesion may be suspected. Improving existing diagnostic techniques or introducing new technologies that enhance assessment parameters, such as accuracy, sensitivity, specificity, or positive predictive value, may assist physicians in detecting breast cancer at an early stage. Early detection may be essential for effective intervention and outcomes in women's health.

Common breast cancer screening methods may include the clinical breast exam (CBE) and X-ray mammography. These techniques may have been questioned due to their relatively low sensitivity. Although X-ray mammography may remain a standard preliminary screening modality, it may be associated with patient discomfort. X-ray mammography may involve the use of ionizing radiation, which may present potential health risks.

In clinical practice, ultrasound, X-ray mammography, and magnetic resonance imaging (MRI) may be widely used for breast cancer detection. X-ray mammography may serve as the primary screening technique. Despite its widespread use, X-ray mammography may be less effective in certain populations, particularly in women under the age of 50 and those with dense breast tissue. X-ray mammography may demonstrate a sensitivity of only 67.8%. In addition to its limited performance in these groups, mammography's reliance on ionizing radiation may further underscore the need for alternative, non-ionizing imaging approaches. Consequently, other modalities, such as diffuse optical tomography, impedance spectroscopy, and elastography may be being explored as potential adjuncts or replacements.

Over the past three decades, substantial advances in optoelectronics and fiber optics may have contributed to the rapid development of near-infrared (NIR) optical imaging techniques. NIR optical imaging, when integrated with optoelectrical devices and fiber optics, may have emerged as a noninvasive and promising method for functional and compositional imaging of highly scattering biological tissues, including the breast and brain.

The optical properties of the breast tissue may have been extensively studied. It may be well established that the main chromophores in breast tissue, namely water, fat, deoxyhemoglobin (Hb), and oxyhemoglobin (HbO2), may exhibit low absorption in the wavelength range of 650 to 1100 nanometers. This so-called “optical window” may enable deeper light penetration, making it suitable for imaging applications in breast tissue.

Numerous handheld diffuse optical probes may have been developed and tested for breast cancer screening and detection. These devices may be typically configured in reflectance geometry to measure the optical properties of the breast tissue located beneath the probe. Laser or fiber-coupled laser light sources operating in the NIR range may be used to quantify chromophore concentrations associated with tissue abnormalities. However, existing laser and fiber-based systems may be often complex, bulky, and expensive, limiting their applicability in point-of-care or widespread clinical settings.

### BRIEF DESCRIPTION OF THE DRAWINGS

Various aspects and many of the attendant advantages of the subject matter disclosed herein will become more readily appreciated as the same becomes better understood by reference to the following detailed description, when taken in conjunction with the accompanying drawings.

FIG. 1 shows a handheld probe for use in an improved human tissue imaging system according to an embodiment of the subject matter disclosed herein.

FIG. 2 shows computing system for use in an improved human tissue imaging system according to an embodiment of the subject matter disclosed herein.

FIG. 3 shows a graphical user interface with graphs with various lines showing data trends for wavelengths 670 nm, 810 nm, and 905 nm for use in an improved human tissue imaging system according to an embodiment of the subject matter disclosed herein.

FIG. 4 shows a close-up view of the handheld probe of FIG. 1 for use in an improved human tissue imaging system according to an embodiment of the subject matter disclosed herein.

FIG. 5 shows a block diagram illustrating the components of an improved handheld probe system for imaging human tissue according to an embodiment of the subject matter disclosed herein.

FIG. 6 shows a schematic of the signal processing system for a handheld probe according to an embodiment of the subject matter disclosed herein.

FIG. 7 shows absorption coefficients for a solid phantom with an abnormality (a) in the center, (b) offset to LED1 (left), and (c) offset to LED2 (right) according to an embodiment of the subject matter disclosed herein.

FIG. 8 shows absorption coefficients for three different example patients corresponding to the examples of FIG. 7 according to an embodiment of the subject matter disclosed herein.

FIG. 9 shows a diagrammatic representation of algorithmic calculations for signals from two LED sources labeled eLED #1 and eLED #2 at positions (0,0) and (x2,y2) according to an embodiment of the subject matter disclosed herein.

FIG. 10 shows a diagrammatic representation of algorithmic calculations for signals from two eLEDs labeled as eLED #1 and eLED #2 on each side of a central axis according to an embodiment of the subject matter disclosed herein.

FIG. 11 shows four exemplary heat map images displaying data on the length and depth of tissue corresponding to the approach of FIG. 10 according to an embodiment of the subject matter disclosed herein.

FIG. 12 shows a diagrammatic representation of algorithmic calculations for signals from two LEDs labeled as eLED #1 and eLED #2 above a central axis according to an embodiment of the subject matter disclosed herein.

FIG. 13 shows three exemplary heat map images displaying data on length and depth of tissue corresponding to the approach of FIG. 12 according to an embodiment of the subject matter disclosed herein.

FIG. 14 shows exemplary tumor location diagrams having cross-sectional views with tumors marked, ultrasound images displaying portions of human tissue, and false-color images representing probe data with varying color gradients indicating different data points according to an embodiment of the subject matter disclosed herein.

FIG. 15 shows a grid of eight exemplary 3D color-coded plots labeled “Slices 1 to 8” showing variations in data across axes labeled X, Y, Z according to an embodiment of the subject matter disclosed herein.

FIG. 16 shows a set of sixteen exemplary 3D color-coded plots divided into two groups according to an embodiment of the subject matter disclosed herein.

FIG. 17 shows exemplary 3D graphs with layers of colorful contours, transitioning from blue to red, representing patient data according to an embodiment of the subject matter disclosed herein.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the subject matter disclosed herein. This disclosure is not intended to be limited to the embodiments shown but is to be accorded the widest scope consistent with the principles and features disclosed or suggested herein.

By way of an overview, the system may include a handheld probe configured to emit optoelectrical energy, specifically near-infrared light waves, at wavelengths corresponding to the absorption characteristics of human tissue constituents. The probe may also detect the returned optoelectrical energy at various wavelengths. An electronic assembly may process this data to generate cross-sectional images of human tissue. The system may further include a computing device executing software to analyze, assemble and present the data and adjust the operational parameters of the handheld probe.

The handheld probe may include sensors and emitters aligned along a common axis with orientation outward. Light-emitting sources, such as LEDs, may be positioned on either side of the sensors and may emit in the visible, near-infrared, and infrared spectra. These wavelengths may correspond to absorption characteristics of tissue constituents such as deoxyhemoglobin, oxyhemoglobin, water, and fat. While these embodiments of particular wavelengths (e.g., visible, near-infrared, and infrared spectra) are disclosed further herein, other wavelengths are also possible which are suited for additional tissue constituents for detection other than constituents such as deoxyhemoglobin, oxyhemoglobin, water, and fat.

A method for scanning human tissue using diffuse optical spectroscopy may include emitting optoelectrical energy at wavelengths corresponding to tissue chromophores, detecting returned energy with the handheld probe, and using a processor to generate cross-sectional images. Sensors may be aligned along a first axis and facing outward, with emitters positioned on either side. Emitted wavelengths may span the visible to infrared spectrum, targeting tissue constituents such as deoxyhemoglobin, oxyhemoglobin, water, and fat.

In another embodiment, the system may comprise a light source for emitting optoelectrical energy at tissue-specific wavelengths, a receiver for detecting the returned signal, and a processor for generating cross-sectional tissue images based on the received data.

Diffuse optical spectroscopy (DOS) and diffuse optical imaging (DOI) may be noninvasive and non-ionizing techniques for breast cancer diagnosis. The handheld probe device may measure the optical properties of breast tissue to generate functional and compositional images of the tissue. Encapsulated Multi-wavelength (LEDs or eLEDSs), disposed in an encapsulated package, may illuminate the tissue, while a linear imaging sensor may capture the photon intensities at various radial distances from the illumination source. This configuration may allow for device miniaturization while maintaining high reliability and imaging accuracy. Multi-wavelength illumination may enable imaging of specific chromophores, including deoxyhemoglobin, oxyhemoglobin, fat, and water. The device may also measure the average scattering.

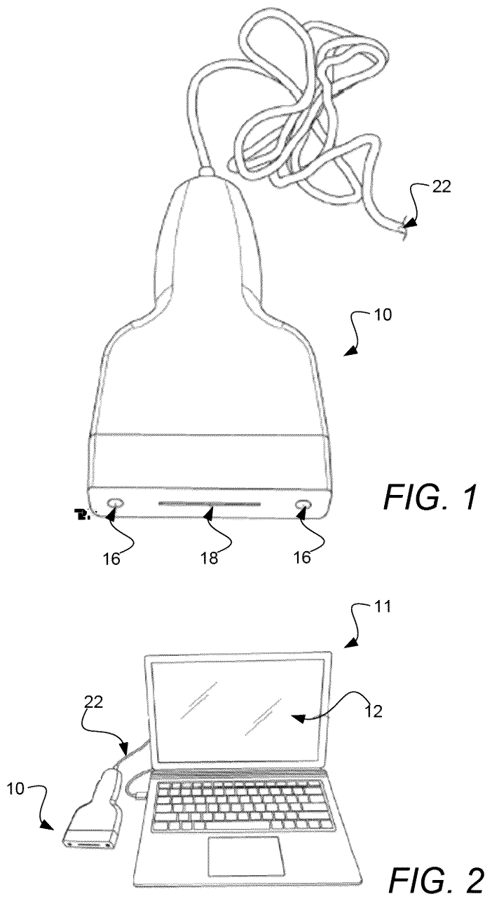

A more detailed embodiment of the breast cancer imaging probe may be demonstrated in conjunction with FIGS. 1-17. This version may represent a significant improvement over a prior version disclosed in U.S. Pat. No. 11,457,818B2 (A1), which is incorporated herein by reference in its entirety. The handheld probe may integrate enhanced components and configurations to improve imaging depth, accuracy, and usability. It may enable real-time, high-resolution imaging suitable for effective breast cancer diagnostics. The system may be designed to display the composition of at least two tissue constituents, such as deoxyhemoglobin, oxyhemoglobin, water, fat, bone, or the like, on a gradient color scale. This may facilitate the detection of tumors, other anomalies, and inflammatory conditions such as arthritis. A modular head may enable the system to be adapted for various applications, including but not limited to breast cancer, skin cancer, colon cancer, rheumatoid arthritis, and imaging of organs such as the thyroid, liver, and kidneys. In each application, the system may identify variations in tissue composition based on at least two biological constituents.

For certain applications such as rheumatoid arthritis, the imaging sensor and at least one emitter may be positioned facing each other on opposing axes to evaluate tissue between them. This configuration may supplement coaxial emitter placement aligned with the imaging sensor.

The handheld probe may be designed to improve breast cancer imaging through real-time, high-resolution optical imaging. Its integrated components may enable precise detection and diagnostic accuracy for breast tissue abnormalities.

FIG. 1 shows a handheld probe 10 for use in an improved human tissue imaging system according to an embodiment of the subject matter disclosed herein. The handheld probe 10 may comprise a housing configured to be held and manipulated by a user during tissue scanning operations. The probe cable 22 may extend from the handheld probe 10 to provide an interface for electrical signal communication between the probe components and external processing equipment. The probe cable 22 may carry power signals, control signals, and data signals between the handheld probe 10 and a connected computing device. Communication protocols for the probe 10 via the probe cable 22 (or otherwise) may be USB 2.0 or any other suitable communication protocol including wireless and near field communication.

The handheld probe 10 may include multiple light emitters 16 positioned within the housing. The light emitters 16 may be configured to emit electromagnetic radiation at specific wavelengths corresponding to absorption characteristics of human tissue constituents. The light emitters 16 may comprise light-emitting diodes, encapsulated light-emitting diodes, or laser diodes capable of emitting near-infrared radiation. The light emitters 16 may be positioned to direct emitted radiation outward from the handheld probe 10 toward target tissue during scanning operations.

The handheld probe 10 may further include a light sensor 18 configured to detect electromagnetic radiation reflected or scattered from tissue. The light sensor 18 may be positioned between or adjacent to the light emitters 16 within the housing of the handheld probe 10. In other embodiments not pictured, the light sensor 18 may be positioned facing and/or opposite the light emitters 16. The light sensor 18 may comprise a charge-coupled device, a complementary metal-oxide-semiconductor imaging sensor, a linear photodiode array, or other suitable detector technology. The light sensor 18 may be aligned along a common axis with the light emitters 16 and oriented to face outward from the handheld probe 10.

The light emitters 16 and light sensor 18 may be arranged in a reflectance geometry configuration within the handheld probe 10. The light emitters 16 may be positioned equidistant from the light sensor 18 to provide balanced illumination of target tissue. The spacing between the light emitters 16 and the light sensor 18 may be optimized to achieve desired penetration depth and signal quality for the intended tissue imaging application.

FIG. 2 shows computing system for use in an improved human tissue imaging system according to an embodiment of the subject matter disclosed herein. A computing device 11 may comprise a laptop computer, desktop computer, tablet computer, or other suitable computing platform configured to communicate with and control the handheld probe 10. The computing device 11 may include a processor, memory, and input/output interfaces necessary for executing software applications and managing data communication with the handheld probe 10. The display 12 may be integrated with the computing device 11 or may comprise a separate monitor or display screen configured to present visual information to a user.

The probe cable 22 may provide a physical communication link between the handheld probe 10 and the computing device 11. The probe cable 22 may carry electrical signals including power supply lines, control signals, and data transmission lines. The probe cable 22 may comprise a multi-conductor cable with appropriate shielding to minimize electromagnetic interference and ensure reliable signal transmission between the handheld probe 10 and the computing device 11. The probe cable 22 may include standard connectors such as USB, Ethernet, or proprietary connectors designed specifically for the tissue imaging system. In other embodiments not shown, the handheld probe 10 may be communicatively coupled to the computing device using common wireless methods, such as Wi-Fi or Blue Tooth™.

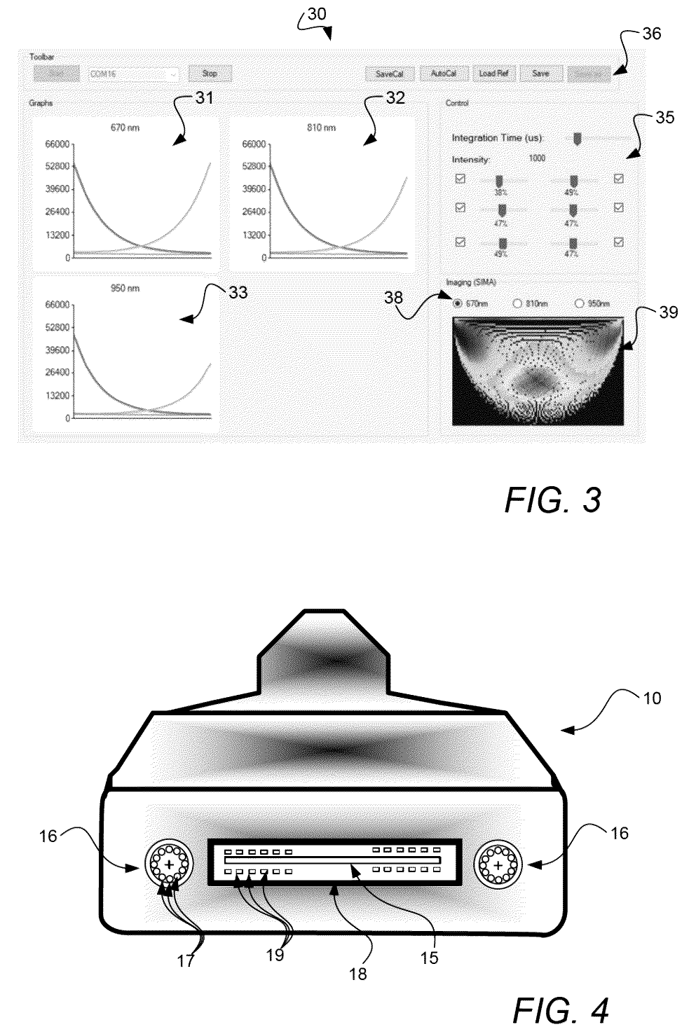

The computing device 11 may execute specialized software applications configured to control the operation of the handheld probe 10. The software may provide user interface controls (shown below with respect to FIG. 3) for adjusting operational parameters of the light emitters 16 and light sensor 18 within the handheld probe 10. The computing device 11 may process data received from the handheld probe 10 to generate cross-sectional images of human tissue. The processing may include signal conditioning, noise reduction, and image reconstruction algorithms that convert raw optical data into meaningful visual representations of tissue composition and structure.

The display 12 may present real-time imaging data during tissue scanning operations. The display 12 may show cross-sectional images corresponding to different wavelengths of electromagnetic radiation emitted by the handheld probe 10. The display 12 may provide visual feedback regarding probe positioning, contact pressure, and signal quality to assist a user in obtaining optimal imaging results. The display 12 may also present analytical tools and measurement capabilities for quantifying tissue characteristics and identifying regions of interest within the scanned tissue.

The computing system may support wireless communication protocols as an alternative to the probe cable 22. The handheld probe 10 may include wireless communication modules such as Bluetooth or Wi-Fi transceivers for transmitting data to the computing device 11. The wireless communication capability may enhance user mobility and reduce physical constraints during tissue scanning procedures. The computing device 11 may be configured to receive and process wireless data transmissions from the handheld probe 10 while maintaining real-time imaging performance.

FIG. 3 shows a graphical user interface 30 with graphs with various presentations showing data trends for wavelengths 670 nm, 810 nm, and 905 nm for use in an improved human tissue imaging system according to an embodiment of the subject matter disclosed herein. The software interface 30 may provide comprehensive control and visualization capabilities for the handheld probe system. The software interface 30 may include multiple display components configured to present real-time imaging data and operational parameters to a user during tissue scanning procedures.

The software interface 30 may include a 670 nm display 31 configured to present imaging data corresponding to electromagnetic radiation at the 670 nanometer wavelength. The 670 nm display 31 may show cross-sectional tissue images that correspond to absorption characteristics of specific tissue constituents at this wavelength. The 670 nm display 31 may present visual representations of tissue composition data in real-time as the handheld probe 10 captures reflected electromagnetic radiation.

The software interface 30 may further include an 810 nm display 32 configured to present imaging data corresponding to electromagnetic radiation at the 810 nanometer wavelength. The 810 nm display 32 may show cross-sectional tissue images that reveal different tissue constituent characteristics compared to the 670 nm display 31. The 810 nm display 32 may provide complementary imaging information that enhances the overall tissue analysis capabilities of the system.

The software interface 30 may also include a 960 nm display 33 configured to present imaging data corresponding to electromagnetic radiation at the 960 nanometer wavelength. The 960 nm display 33 may show cross-sectional tissue images that correspond to absorption and scattering characteristics of tissue constituents at this specific wavelength. The 960 nm display 33 may provide additional spectral information that contributes to comprehensive tissue characterization.

The software interface 30 may include control parameters 35 configured to allow user adjustment of operational settings for the handheld probe 10. The control parameters 35 may include settings for integration time, light intensity levels, and acquisition parameters. The control parameters 35 may enable users to optimize imaging conditions based on specific tissue types or scanning requirements.

The software interface 30 may incorporate software navigation controls 36 configured to provide user interface elements for system operation and data management. The software navigation controls 36 may include buttons, menus, and selection tools that allow users to navigate between different imaging modes and analysis functions. The software navigation controls 36 may facilitate efficient operation of the tissue imaging system during clinical procedures.

The software interface 30 may include radial buttons for imaging display 38 configured to provide user selection options for different imaging display modes. The radial buttons for imaging display 38 may allow users to select between different visualization formats or analysis parameters. The radial buttons for imaging display 38 may enable rapid switching between different imaging configurations during tissue scanning operations including views of specific wavelength data.

The software interface 30 may further include an imaging display 39 configured to present processed imaging data in a format suitable for clinical interpretation. The imaging display 39 may show composite images that combine data from multiple wavelengths to provide comprehensive tissue analysis. The imaging display 39 may present color-coded representations of tissue composition and functional characteristics based on the multi-wavelength optical data collected by the handheld probe 10.

FIG. 4 shows a close-up view of the handheld probe of FIG. 1 for use in an improved human tissue imaging system according to an embodiment of the subject matter disclosed herein. It may include two or more multi-wavelength light emitters 16 tailored to tissue absorption spectra, a light sensor 18 to detect reflected optoelectrical signals, and a local controller (not shown in FIG. 4) that controls the light emitters 16 and light sensor 18. The handheld probe 10 may include a light source 16 positioned within the housing to emit electromagnetic radiation toward the target tissue. The light emitters 16 may comprise multiple light source LEDs 17 configured to emit radiation at specific wavelengths corresponding to absorption characteristics of human tissue constituents. The light source LEDs 17 may be arranged in a linear or circular configuration within the handheld probe 10 to provide uniform illumination of the target tissue area. The light source LEDs 17 may emit radiation in the near-infrared spectrum range, including wavelengths such as 670 nanometers, 810 nanometers, and 950 nanometers.

The handheld probe 10 may further include a light sensor 18 configured to detect electromagnetic radiation reflected or scattered from tissue. The light sensor 18 may be positioned adjacent to or between the light emitters 16 within the housing of the handheld probe 10. The light sensor 18 may comprise multiple light sensor CMOS elements 19 arranged in a linear array configuration. The light receiver CMOS elements 19 may be configured to capture photon intensities at various radial distances from the light emitters 16. Each of the light sensor CMOS elements 19 may be optimized for sensitivity in the near-infrared wavelength range to minimize ambient light interference and maximize signal-to-noise ratio.

The light sensor CMOS elements 19 may be positioned along a common axis with the light emitters 16 and oriented to face outward from the handheld probe 10. The spacing between the light source LEDs 17 and the light sensor CMOS elements 19 may be configured to achieve optimal penetration depth and signal quality for tissue imaging applications. The light sensor CMOS elements 19 may provide high-resolution detection capabilities with pixel sizes optimized for the intended imaging depth and resolution requirements.

The light emitters 16 and light sensor 18 may operate in a reflectance geometry configuration within the handheld probe 10. The light source LEDs 17 may emit radiation sequentially or simultaneously at multiple wavelengths, while the light sensor CMOS elements 19 may capture the corresponding reflected radiation for each wavelength. The captured data from the light sensor CMOS elements 19 may be transmitted to processing circuitry for real-time image reconstruction and analysis.

Further, the handheld probe 10 may include one or more sensors 15 to detect probe orientation and contact pressure. The auxiliary sensors 15 may comprise multiple sensor types configured to provide comprehensive contact force and orientation measurement capabilities for the handheld probe 10. The auxiliary sensors 15 may include force-sensitive resistors positioned at contact points between the handheld probe 10 and target tissue. The force-sensitive resistors may be configured to detect variations in applied pressure during tissue scanning operations. The force-sensitive resistors may comprise polymer-based sensors with resistance values that change proportionally to applied force.

The auxiliary sensors 15 may further include accelerometer sensors configured to detect orientation changes of the handheld probe 10 relative to the tissue surface. The accelerometer sensors may comprise three-axis MEMS accelerometers capable of measuring gravitational acceleration components along orthogonal axes. The accelerometer sensors may provide real-time feedback regarding probe tilt angles and positioning stability during scanning procedures.

The auxiliary sensors 15 may also include gyroscopic sensors configured to measure angular velocity and rotational movement of the handheld probe 10. The gyroscopic sensors may comprise three-axis MEMS gyroscopes that detect rotational motion about each axis. The gyroscopic sensors may complement the accelerometer sensors to provide comprehensive orientation tracking capabilities.

The placement locations of the auxiliary sensors 15 may be strategically distributed throughout the handheld probe 10 housing. The force-sensitive resistors may be positioned at the probe tip adjacent to the light emitters 16 and light sensor 18. The force-sensitive resistors may be embedded within the probe housing surface that contacts the tissue during scanning operations. The accelerometer and gyroscopic sensors may be mounted on the main circuit board within the probe housing to minimize mechanical interference.

The integration methods for the auxiliary sensors 15 may involve direct connection to the microcontroller unit within the handheld probe 10. The force-sensitive resistors may be connected through analog-to-digital converter channels of the microcontroller. The accelerometer and gyroscopic sensors may communicate with the microcontroller through digital interfaces such as I2C or SPI protocols. The sensor integration may include appropriate signal conditioning circuitry to ensure accurate measurement and noise reduction.

The calibration procedures for the auxiliary sensors 15 may include factory calibration and field calibration protocols. The force-sensitive resistors may undergo calibration using known reference weights to establish force-to-resistance transfer functions. The accelerometer sensors may be calibrated using gravitational reference orientations to determine offset and scaling parameters. The gyroscopic sensors may require zero-rate offset calibration to compensate for inherent sensor drift characteristics.

The data fusion techniques may combine measurements from multiple auxiliary sensors 15 to provide enhanced contact force and orientation information. The force measurements from multiple force-sensitive resistors may be averaged or weighted to provide overall contact pressure values. The accelerometer and gyroscopic sensor data may be processed through complementary filter algorithms to provide stable orientation estimates. The data fusion algorithms may include Kalman filtering techniques to combine sensor measurements and reduce measurement uncertainty.

The auxiliary sensors 15 may interface with the main imaging system through the microcontroller unit that also controls the light emitters 16 and light sensor 18. The sensor data may be transmitted to the computing device 11 along with the optical imaging data through the probe cable 22 or wireless communication interface. The computing device 11 may process the auxiliary sensor data to provide real-time feedback to the user regarding probe contact quality and positioning accuracy.

The software interface 30 may display auxiliary sensor information alongside the imaging data from the light sensor 18. The contact force measurements may be presented as numerical values or graphical indicators to guide optimal probe positioning. The orientation measurements may be displayed as angular indicators or level displays to assist users in maintaining proper probe alignment during tissue scanning procedures.

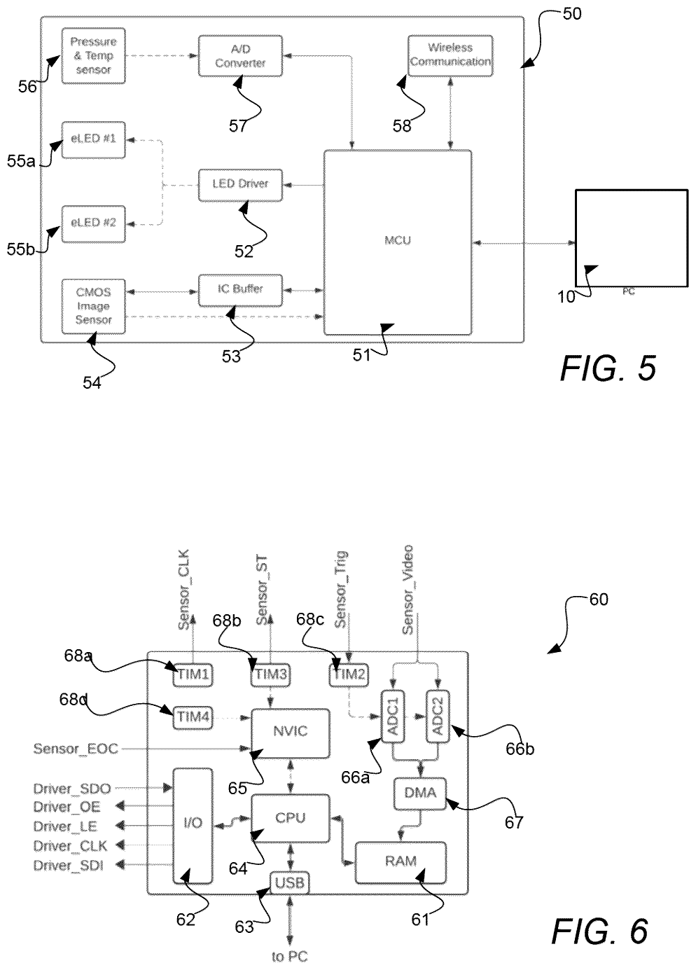

FIG. 5 shows a block diagram 50 of the handheld probe 10 that illustrates the electronic architecture and signal processing components of the improved tissue imaging system. The block diagram 50 may provide a comprehensive overview of the hardware components and their interconnections within the handheld probe 10. An MCU 51 may serve as the central controlling unit that coordinates the operation of all electronic components within the handheld probe. The MCU 51 may execute control algorithms for timing sequences, data acquisition protocols, and communication functions. The MCU 51 may interface with multiple peripheral components to manage the overall system operation during tissue scanning procedures.

An LED Driver 52 may provide controlled electrical power to the light-emitting components within the handheld probe 10. The LED Driver 52 may regulate current levels and switching sequences for the LED components of the light emitters 16 to ensure consistent optical output during multi-wavelength illumination. The LED Driver 52 may receive control signals from the MCU 51 to coordinate the sequential activation of different wavelength sources during scanning operations.

The IC Buffer 53 may provide signal conditioning and amplification functions for the electronic signals within the handheld probe 10. The IC Buffer 53 may isolate sensitive analog signals from digital switching noise and may maintain signal integrity throughout the data acquisition process. The IC Buffer 53 may interface between the CMOS Image Sensor 54 and other processing components to ensure reliable signal transmission.

The CMOS Image Sensor 54 (e.g., CMOS components within the light sensor 18) may capture electromagnetic radiation reflected from tissue during scanning operations. The CMOS Image Sensor 54 may comprise a linear array of photodetectors configured to detect near-infrared radiation at multiple wavelengths. The CMOS Image Sensor 54 may convert optical signals into electrical signals that may be processed by subsequent electronic components within the handheld probe 10.

An eLED #1 55a (e.g., components corresponding to a first light emitter 16) may comprise a first multi-wavelength light source positioned within the handheld probe housing. The eLED #1 55a may emit electromagnetic radiation at specific wavelengths corresponding to absorption characteristics of human tissue constituents. The eLED #1 55a may be controlled by the LED Driver 52 to provide sequential illumination at different wavelengths during tissue scanning procedures.

An eLED #2 55b (e.g., components corresponding to a first light emitter 16) may comprise a second multi-wavelength light source positioned within the handheld probe housing. The eLED #2 55b may emit electromagnetic radiation at wavelengths that may complement those emitted by eLED #1 55a. The eLED #2 55b may be positioned to provide balanced illumination of target tissue in conjunction with eLED #1 55a during scanning operations.

A Pressure and Temperature Sensor 56 may monitor physical contact conditions between the handheld probe and tissue surfaces. The Pressure and Temperature Sensor 56 may provide feedback regarding contact pressure to ensure optimal coupling between the probe and tissue during scanning procedures. The Pressure and Temperature Sensor 56 may also monitor temperature conditions to maintain stable operating parameters for the optical components.

An Analog-to-Digital (A/D) Converter 57 may convert analog electrical signals from the CMOS Image Sensor 54 into digital data formats suitable for processing by the MCU 51. The A/D Converter 57 may provide high-resolution digitization of optical signals to preserve measurement accuracy during data acquisition. The A/D Converter 57 may operate at sampling rates sufficient to support real-time imaging capabilities of the handheld probe system.

The Wireless Communication Unit 58 may enable data transmission between the handheld probe and external computing devices. The Wireless Communication Unit 58 may support wireless protocols such as Bluetooth or Wi-Fi for transmitting digitized imaging data to connected processing systems. The Wireless Communication Unit 58 may provide bidirectional communication capabilities for receiving control commands and transmitting measurement data during tissue scanning operations.

In an embodiment, the handheld device 10 of FIG. 4 may perform compositional and functional cross-sectional imaging of breast tissue using single or multi-wavelength near-infrared LEDs (600-1,000 nm) encapsulated as LEDs within light emitters 16. A 4,096-pixel light sensor 18 (e.g., linear imaging sensor) may measure the scattered or reflected photon intensity to generate images of breast tissue. Selected wavelengths may correspond to absorption profiles of tissue constituents.

The system may generate, in vivo, two-dimensional cross-sectional images by aligning light emitters 16 on either side of the light sensor 18 to maximize photon diffusion, especially in low-fat tissue such as the breast. Using a configuration with three eLED wavelengths, the device may sequentially illuminate tissue, capture scattered or reflected light and reconstruct four compositional images per cycle. Chromophore-specific absorption (e.g., Hb, HbO2) may be calculated and processed in real-time at 24 frames per second (fps), with a capability of up to 60 fps.

The host computer 20 (FIG. 2) may contain a processor and memory and may communicate with the probe 10 via USB, Bluetooth or Wi-Fi. The system may include eight eLED sources 17 within the light emitters 16, power and battery units, and a wireless communication module. The light sensor 18 may capture each illuminated wavelength and may transmit digitized data via a microcontroller (MCU), which may control the light emitters 16 and light sensor 18 functions.

Two light emitters 16 positioned at each end of the light sensor 18 may emit light at 670 nm, 810 nm, and 950 nm to optimize photon penetration in low-fat breast tissue. The light emitters 16 may be triggered individually or sequentially at 200 ms intervals. The light sensor 18 (e.g., a S19364 CMOS array) may feature 4,096 closely spaced pixels over a 28.672 mm active area, with a photodetector circuit designed to accommodate variable light intensities due to source-detector spacing.

NIR photon behavior in tissue may follow diffuse optical spectroscopy principles, with Hb, HbO2, water, and fat as primary absorbers. This may enable deep tissue penetration. Data collected at three wavelengths may yield 24,576 measurement points per frame. Light transport may be modeled using the diffusion equation, and absorption coefficients may be used to construct cross-sectional images.

A software interface compatible with Python, MATLAB, and C (or other suitable programming language) may provide full control over hardware, data collection, and imaging functions. Communication may be supported via USB 2.0 or similar protocols, with a graphical user interface (GUI) (FIG. 3) for source selection, parameter adjustment, and real-time data capture.

The handheld probe 10 may include several operational improvements to enhance usability and diagnostic accuracy. Real-Time Imaging may provide immediate feedback crucial for timely diagnosis and treatment planning. Advanced Measurement Algorithms may facilitate the detection of depth-related information.

The handheld probe 10 may include specific hardware enhancements and differences with respect to predecessors. In a previous design, the ILX511 Linear CCD sensor may have been used as an optical detector and multi-wavelength NIR LED with limited effective penetration depth as the light source. One conventional problem in conventional handheld probes may have been unstable performance in real-time imaging, low computation capacity on the processor, and visible light noise due to the sensor's improper sensitivity spectrum. In the improved handheld probe 10, all the components may be improved to fix these problems while maintaining the general aim of the design as a NIR multi-wavelength linear handheld probe. These improvements are detailed next.

Improved Light Emitter: The light emitters 16 may be chosen to be multi-wavelength NIR LEDs positioned along the axis of the light sensor 18. The light emitters 16 (MTMD6894T38) may be a multi-chip emitter designed for applications requiring various emission sources in a small, densely packed area. It may contain two or more LED units 17 for each wavelength (670, 810, 950 nm), maximizing the tissue's output power and penetration depth. As tested in the lab, the light emitters 16 may be powerful enough to be employed in rheumatoid arthritis applications for different joints and skin colors in reflection and transmission design.

Improved Light Sensor: The light sensor 18 may be an Enhanced Optical Sensor Array (Hamamatsu S13496) which is a CMOS linear image sensor consisting of 4096 pixels, each with a pixel size of 7×200 micro-meter. This light sensor 18 may have high sensitivity in the NIR region and low response in the visible range, reducing ambient noise and resulting in better SNR. The light sensor 18 may still have a fully serial analog output while exhibiting twice as many pixels. The maximum video data rate may be 10 MHz, which is five times more than conventional handheld probes. It may also have three times more photosensitivity in the peak response; for the new sensor, it may actually fall in the NIR range.

In conventional handheld probe, a user may have set the integration time in the 1800-20000 micro-second range, and then the number of frames captured and averaged for each light pulse may have been selected based on that. Shorter integration time on the light sensor may have led to more frames per pulse. The logic behind this pattern may have been to replace the averaging operation with the summation, which had benefits by having a lower processing load and 8-bit ADC output than 16-bit, but with the cost of losing precision. Also, this led to variable output frames per second (fps) in real-time imaging, which may have been unnecessary and problematic for the host computer.

In the handheld probe 10 of FIG. 4-5, output fps may be kept constant to help stabilize plotting and capturing in the GUI. Also, because of having a much faster and more sensitive light sensor 18, there may be a wider range for integration time containing low values, such as 500 microseconds. Four first captured frames may be averaged for each optical pulse, resulting in a 4096-pixel frame with high Signal-to-Noise ratios. The user may choose to have the full resolution or binned output.

Another difference in the improved handheld probe 10 architecture is that a better core is utilized taking advantage of NIR-sensitive CMOS light sensor 18, and internal A/DCs instead of a separate A/DC IC. One advantage of having internal A/DC is that it can benefit from direct A/DC and connections through the bus matrix. Also, there is no need to create controlling and communication signals with external A/DC, but it may have a slightly lower accuracy.

To compensate for a lower sampling rate on internal A/DCs, two independent A/DCs may be used with external triggers coming from the light sensor 18, triggering the A/DCs in rising and falling edges alternatively to guarantee precise timing of conversions. In this processor, each A/DC may be capable of having a maximum 5 MHz sampling rate in 12-bit resolution and can capture a frame in the light sensor's max clock.

FIG. 5 may show the handheld probe 10 hardware that builds upon the foundation conventional hardware, incorporating significant enhancements in light source efficiency, detector sensitivity, processing power, and overall system integration. These improvements aim to provide more accurate, reliable, and user-friendly imaging capabilities, addressing limitations of previous generations of hardware and enhancing the overall effectiveness of the improved handheld probe 10 in breast cancer diagnostics and treatment monitoring.

FIG. 6 shows a detailed schematic diagram of the probe IC 60 that may serve as the central processing and control unit for the handheld probe 10. The probe IC 60 may comprise multiple integrated components configured to manage data acquisition, signal processing, and communication functions within the handheld probe device. The probe IC 60 may be implemented as a microcontroller unit or system-on-chip architecture that coordinates the operation of all electronic subsystems within the handheld probe.

Onboard RAM 61 may provide volatile memory storage for temporary data buffering and real-time processing operations within the probe IC 60. The RAM 61 may store light sensor data, intermediate processing results, and operational parameters during tissue scanning procedures. The RAM 61 may be configured with sufficient capacity to support high-speed data acquisition and processing requirements of the multi-wavelength optical imaging system.

The Input/Output integrated circuit 62 may manage digital signal interfaces and communication protocols between the probe IC 60 and external components. The Input/Output IC 62 may handle control signals for the light emitters, light sensor interfaces, and peripheral device communications. The Input/Output IC 62 may provide signal conditioning and level translation functions to ensure compatibility between different electronic subsystems within the handheld probe 10.

AUSB port 63 may provide a standardized communication interface for data transfer between the probe IC 60 and external computing devices. The USB port 63 may support high-speed data transmission of acquired imaging data to connected processing systems. The USB port 63 may also provide power delivery capabilities for charging internal battery systems or powering the probe IC 60 during operation.

A CPU integrated circuit 64 may serve as the primary processing unit within the probe IC 60, executing control algorithms and data processing functions. The CPU 64 may coordinate the timing sequences for multi-wavelength illumination and light sensor data acquisition. The CPU 64 may implement signal processing algorithms for noise reduction, calibration corrections, and preliminary data analysis before transmission to external computing systems.

The NVIC 65 may provide nested vectored interrupt control functionality for managing multiple simultaneous interrupt sources within the probe IC 60. The NVIC 65 may prioritize and handle interrupt requests from various subsystems including timers, analog-to-digital converters, and communication interfaces. The NVIC 65 may ensure deterministic response times for time-critical operations such as light sensor data acquisition and light emitter control.

An ADC1 66a may comprise a first on-chip analog-to-digital converter configured to digitize analog signals from the light sensor array. The ADC1 66a may provide high-resolution conversion of light sensor outputs to digital data formats suitable for processing by the CPU 64. The ADC1 66a may operate at sufficient sampling rates to capture rapid changes in optical signals during multi-wavelength scanning procedures.

The ADC2 66b may comprise a second on-chip analog-to-digital converter that may provide additional conversion capacity for simultaneous multi-channel data acquisition. The ADC2 66b may be configured to work in conjunction with ADC1 66a to increase the overall system throughput and reduce conversion latency. The ADC2 66b may be triggered alternately with ADC1 66a to achieve higher effective sampling rates for time-critical measurements.

A DMA 67 may provide direct memory access functionality for high-speed data transfer between peripheral devices and memory without CPU intervention. The DMA 67 may manage the transfer of digitized light sensor data from the analog-to-digital converters to RAM 61 storage locations. The DMA 67 may reduce CPU processing overhead and enable continuous data acquisition during intensive processing operations.

A series of four timer modules 68a-68d may be utilized to facilitate functionality of the probe IC 60. A TIM1 68a may comprise a first timer module configured to generate precise timing signals for sensor clock generation. The TIM1 68a may produce pulse-width modulated signals with controlled duty cycles for driving the optical light sensor array. The TIM1 68a may provide the master timing reference for coordinating data acquisition sequences across multiple system components.

A TIM2 68b may comprise a second timer module that may be configured for light source control and sequencing operations. The TIM2 68b may generate timing signals for coordinating the sequential activation of different wavelength light emitters during scanning procedures. The TIM2 68b may provide precise timing control for optimizing light exposure durations and minimizing crosstalk between different wavelength measurements.

A TIM3 68c may comprise a third timer module that may generate sensor trigger signals for initiating data acquisition cycles. The TIM3 68c may produce start pulses that define the integration time for optical light sensor measurements. The TIM3 68c may coordinate with other timer modules to ensure synchronized operation of the entire data acquisition system.

A TIM4 68d may comprise a fourth timer module that may be configured for frame rate control and system timing coordination. The TIM4 68d may operate in interrupt mode to provide periodic timing references for maintaining consistent data acquisition rates. The TIM4 68d may coordinate with the CPU 64 to manage overall system timing and ensure stable real-time imaging performance.

TIM1 68a may generate the sensor clock as a PWM signal with a 50% duty cycle. TIM4 68d, with no output in interrupt mode, may be used for timing purposes of light pulses and fps. The LED driver may be programmed to switch to the next wavelength and set the corresponding intensity in its callback function. TIM3 68c may generate the ST pulse to start frame capture in the light sensor 18. The high period of the ST pulse may determine the integration time. Another channel of TIM3 68c may be set to interrupt the CPU at the right time to start the ADCs in DMA mode. The end of the conversion interrupt from ADC may call the callback function to execute the pixel-wise and frame-wise averaging. The light sensor 18 may generate an EOC (end of capture) pulse to indicate the end of the frame output stream and may be used as an external interrupt to send out the final frame through USB communication.

The handheld probe 10 may be configured to operate at frame rates up to 60 frames per second through several coordinated hardware and software optimizations. The enhanced frame rate capability may be achieved through the implementation of a high-performance ARM Cortex-M7 processor running at 550 MHz within the MCU 51. The increased processing power may enable rapid data acquisition cycles and real-time signal processing operations necessary for sustained high-speed imaging.

The light sensor 18 may comprise a Hamamatsu S13496 CMOS linear image sensor featuring 4096 pixels with enhanced sensitivity characteristics in the near-infrared spectrum. The light sensor 18 may operate with a maximum video data rate of 10 MHz, providing five times the data throughput compared to conventional sensors. The increased data rate may enable the capture of multiple frames within each illumination cycle while maintaining signal quality and measurement accuracy.

The frame rate optimization may be implemented through precise timing control using multiple timer modules within the probe IC 60. The TIM1 68a may generate sensor clock signals as pulse-width modulated signals with 50% duty cycles to synchronize data acquisition operations. The TIM4 68d may operate in interrupt mode to provide periodic timing references for maintaining consistent frame rates during real-time imaging procedures. The coordinated operation of the timer modules may ensure stable temporal synchronization between light emission sequences and sensor data capture cycles.

The data acquisition architecture may utilize dual analog-to-digital converters ADC1 66a and ADC2 66b operating in alternating trigger modes to achieve higher effective sampling rates. The ADC1 66a and ADC2 66b may each operate at maximum sampling rates of 5 MHz with 12-bit resolution, enabling rapid conversion of analog sensor signals to digital data formats. The alternating trigger configuration may be controlled by external trigger signals from the light sensor 18, with rising and falling edge triggers activating the converters in sequence to guarantee precise timing of conversion operations.

The DMA 67 may provide direct memory access functionality to transfer digitized sensor data from the analog-to-digital converters to RAM 61 storage locations without CPU intervention. The DMA 67 may operate continuously during data acquisition cycles to maintain high-speed data transfer rates while reducing processing overhead on the CPU 64. The direct memory access capability may enable sustained data throughput rates necessary for 60 fps operation without interrupting real-time processing functions.

The frame rate control algorithm may implement constant output frame rates to stabilize data visualization and capture operations in the software interface 30. The constant frame rate approach may differ from variable frame rate systems that adjust timing based on integration time settings. The handheld probe 10 may maintain consistent 60 fps output regardless of integration time variations, providing stable real-time imaging performance for clinical applications.

The handheld probe 10 may be configured to operate in transmission-mode imaging configurations for specific clinical applications where enhanced tissue penetration and bilateral assessment capabilities may be required. The transmission-mode configuration may provide complementary imaging information to the reflectance-mode operation previously described, enabling comprehensive tissue characterization through multiple optical interrogation approaches.

The transmission-mode optical configuration may require precise positioning of light sources and detectors to achieve optimal photon transmission through target tissue. The light emitters 16 may be positioned on opposing sides of the target tissue volume to enable direct transmission measurements. The first light emitter 16 may be positioned on one side of the tissue, while the light sensor 18 may be positioned on the opposite side to detect transmitted electromagnetic radiation. This configuration may allow photons to traverse the entire tissue thickness, providing depth-integrated optical property measurements.

The optical path specifications for transmission-mode imaging may differ significantly from reflectance-mode configurations. The source-detector separation distance may be determined by the tissue thickness being examined, with typical separations ranging from 20 millimeters to 100 millimeters depending on the anatomical site. The light emitters 16 may be aligned along an axis perpendicular to the tissue surface to maximize photon transmission efficiency. The light sensor 18 may be positioned directly opposite to the light emitters 16 to capture the maximum number of transmitted photons.

The light source positioning for transmission-mode operation may utilize the same multi-wavelength light emitters 16 described previously, but with modified optical coupling arrangements. The light emitters 16 may be configured with collimating optics to focus the emitted radiation into a narrow beam that may penetrate through tissue with minimal lateral spreading. The collimation may be achieved through the use of optical lenses or fiber optic coupling elements integrated within the handheld probe 10 housing. The beam diameter at the tissue surface may be controlled to optimize the balance between photon flux density and spatial resolution.

The detector arrangements for transmission-mode imaging may employ the same light sensor 18 components but with modified sensitivity settings to accommodate the reduced photon flux inherent in transmission measurements. The light sensor CMOS elements 19 may be configured with extended integration times to accumulate sufficient photon counts for accurate signal-to-noise ratio measurements. The detector aperture may be optimized to collect transmitted photons across a defined solid angle while minimizing the detection of scattered photons that may degrade measurement accuracy.

The optical path length calculations for transmission-mode measurements may account for the direct photon path through tissue as well as the contribution from multiply scattered photons. The mean photon path length may be calculated using Monte Carlo simulation methods that model photon transport through the specific tissue geometry being examined. The path length distribution may be weighted according to the probability of photon detection at the sensor location, enabling accurate absorption coefficient calculations.

The transmission-mode data processing algorithms may differ from reflectance-mode processing due to the distinct photon transport characteristics. The Beer-Lambert law may be applied directly to transmission measurements to calculate absorption coefficients, with corrections applied for scattering effects. The transmitted light intensity may be measured at each wavelength and compared to the incident intensity to determine the total attenuation through the tissue volume. The attenuation measurements may be processed using logarithmic transformation to linearize the relationship between optical properties and measured signals.

The image reconstruction algorithms for transmission-mode data may employ tomographic reconstruction techniques to generate cross-sectional images from multiple projection measurements. The handheld probe 10 may be positioned at multiple locations around the tissue volume to acquire transmission measurements from different angles. The projection data may be processed using filtered back-projection algorithms or iterative reconstruction methods to generate two-dimensional absorption coefficient maps. The reconstruction process may incorporate prior knowledge of tissue anatomy to improve image quality and reduce artifacts.

The transmission-mode imaging may be particularly suitable for applications involving thin tissue structures or anatomical regions where bilateral access may be available. Joint imaging for rheumatoid arthritis assessment may benefit from transmission-mode measurements, where the probe components may be positioned on opposite sides of finger joints or wrist structures. The transmission configuration may enable direct measurement of synovial fluid optical properties and joint space characteristics that may be difficult to assess using reflectance-mode imaging alone.

The data acquisition protocols for transmission-mode operation may require synchronized control of light emission and detection timing. The MCU 51 may coordinate the sequential activation of light emitters 16 on one side of the tissue while simultaneously triggering data acquisition from the light sensor 18 on the opposite side. The timing synchronization may be critical to ensure that transmitted photons are detected during the appropriate integration period and that ambient light interference may be minimized.

The signal processing for transmission-mode measurements may incorporate noise reduction algorithms specifically designed for low-light-level detection. The transmitted photon flux may be significantly lower than reflected flux in reflectance-mode measurements, requiring enhanced signal processing techniques to maintain measurement accuracy. Digital filtering methods may be applied to reduce electronic noise, while averaging techniques may be used to improve signal-to-noise ratios. The processing algorithms may also compensate for variations in source intensity and detector sensitivity across different wavelengths.

The calibration procedures for transmission-mode imaging may involve measurements through reference phantoms with known optical properties. The calibration phantoms may simulate the optical characteristics of target tissues while providing stable reference standards for system validation. The calibration measurements may be used to determine system response functions and to correct for instrumental variations that may affect measurement accuracy. The calibration data may be stored within the handheld probe 10 memory and applied automatically during data processing operations.

The system may further include longitudinal tracking capabilities configured to monitor tissue changes over time and quantify treatment response through systematic comparison of optical property measurements across multiple imaging sessions. The longitudinal tracking module may establish baseline measurements during initial tissue assessment procedures and may implement change detection algorithms to identify significant variations in tissue composition and functional characteristics during subsequent imaging sessions.

The baseline establishment procedures may involve capturing comprehensive optical property maps during the first imaging session to serve as reference standards for future comparisons. The baseline measurements may include absorption coefficient values, scattering coefficient distributions, and chromophore concentration maps for each wavelength used in the multi-spectral imaging protocol. The baseline data may be stored in memory associated with the computing device 11 and may be indexed according to patient identification parameters and anatomical location coordinates to enable accurate retrieval during follow-up imaging sessions.

The longitudinal tracking module may implement data comparison techniques that analyze temporal changes in optical properties by calculating difference maps between current measurements and stored baseline values. The comparison algorithms may compute pixel-wise differences in absorption coefficients across all wavelengths to identify regions where tissue optical properties have changed significantly over time. The difference calculations may account for measurement variability and may apply statistical thresholds to distinguish meaningful changes from normal measurement fluctuations.

Change detection algorithms may be configured to identify statistically significant variations in tissue optical properties that exceed predetermined threshold values. The change detection processing may apply temporal filtering techniques to reduce noise artifacts and may implement spatial correlation analysis to identify coherent regions of change that correspond to physiological alterations in tissue structure or composition. The algorithms may calculate change magnitude metrics that quantify the degree of optical property variation between imaging sessions.

The system may implement quantitative metrics for assessing treatment response based on changes in chromophore concentrations and optical property distributions over time. Treatment response metrics may include percentage changes in tumor-associated absorption coefficients, variations in oxygenation parameters derived from hemoglobin measurements, and alterations in tissue scattering properties that may indicate structural changes following therapeutic intervention. The quantitative assessment may generate numerical scores that correlate with treatment efficacy and may provide objective measures for clinical decision-making.

The longitudinal tracking functionality may include automated region-of-interest identification algorithms that locate and track specific tissue areas across multiple imaging sessions. The region tracking may compensate for patient positioning variations and probe placement differences between sessions by implementing image registration techniques that align corresponding tissue areas. The registration algorithms may use anatomical landmarks and optical property patterns to establish spatial correspondence between measurements acquired at different time points.

The system may calculate residual cancer burden metrics by analyzing changes in abnormal tissue volumes and optical property characteristics following treatment interventions. The residual burden calculations may integrate absorption coefficient changes across three-dimensional tissue volumes and may quantify the reduction in tumor-associated optical signatures over time. The residual cancer burden assessment may provide quantitative measures of treatment effectiveness that complement conventional imaging modalities.

The longitudinal tracking module may implement trend analysis algorithms that identify patterns in optical property changes over extended monitoring periods. The trend analysis may detect gradual changes in tissue composition that occur over weeks or months and may distinguish between treatment-related improvements and disease progression patterns. The trending algorithms may generate predictive models that estimate future tissue changes based on observed temporal patterns in optical property measurements.

The system may provide visualization tools for displaying longitudinal changes in tissue optical properties through time-series plots and comparative image displays. The visualization interface may present side-by-side comparisons of optical property maps acquired at different time points and may highlight regions where significant changes have occurred. The temporal visualization may include color-coded change maps that indicate the magnitude and direction of optical property variations over time.

The quantitative treatment response monitoring may include automated alert generation when optical property changes exceed clinically significant thresholds. The alert system may notify clinicians when measurements indicate potential treatment failure or unexpected tissue changes that warrant further clinical evaluation. The automated monitoring may reduce the burden of manual data interpretation and may enable timely clinical interventions based on objective optical measurements.

The longitudinal tracking system may maintain comprehensive databases of patient-specific optical property measurements that enable population-level analysis of treatment response patterns. The database functionality may support research applications by providing anonymized datasets for developing improved treatment response prediction models and for validating the correlation between optical property changes and clinical outcomes.

The multilayer printed circuit board (PCB) architecture may be specifically designed to address electromagnetic interference challenges inherent in high-frequency optical imaging systems. The PCB design may incorporate multiple ground planes positioned strategically throughout the board stackup to provide low impedance return paths for high-speed digital signals. The ground planes may be implemented as continuous copper layers that may minimize ground bounce and reduce electromagnetic emissions from switching circuits within the handheld probe 10.

The PCB stackup configuration may utilize alternating signal and ground layers to maintain controlled impedance characteristics for critical signal traces. High-speed digital signals from the CMOS Image Sensor 54 may be routed on dedicated signal layers positioned adjacent to ground planes to ensure proper signal integrity.

Power distribution networks within the PCB may be designed with dedicated power planes that may provide stable voltage references for analog and digital circuits. Separate power planes may be implemented for analog circuits, digital circuits, and high-current LED driver circuits to prevent cross-coupling of noise between different functional blocks. The power planes may incorporate copper pour techniques with adequate trace widths to minimize voltage drops and provide sufficient current carrying capacity for peak power demands.

The PCB layout may implement strict separation between analog and digital circuit sections to prevent digital switching noise from interfering with sensitive analog measurements. Analog circuits including the CMOS Image Sensor 54 and associated signal conditioning circuits may be positioned on dedicated areas of the PCB with separate ground regions. Digital circuits including the MCU 51 and communication interfaces may be located in separate PCB regions with their own ground reference areas.

Grounding schemes within the PCB may utilize a star grounding topology where sensitive analog circuits may be connected to a central analog ground point. The analog ground may be connected to the digital ground through a single point connection or ferrite bead to minimize ground loop currents while maintaining a common reference potential. The ground connection strategy may prevent high-frequency digital currents from flowing through analog ground paths and corrupting sensitive optical measurements.

Component placement strategies may position noise-sensitive circuits away from high-frequency switching circuits and electromagnetic interference sources. The CMOS Image Sensor 54 may be positioned at maximum distance from the MCU 51 and LED Driver 52 circuits to minimize electromagnetic coupling. Crystal oscillators and clock generation circuits may be located away from analog signal paths and may be surrounded by ground guard traces to contain electromagnetic emissions.

The PCB may incorporate electromagnetic shielding techniques including ground guard traces around sensitive signal paths and critical analog circuits. Guard traces may be connected to ground potential and may surround high-impedance analog signals to provide electromagnetic shielding. The guard traces may be positioned on the same layer as the protected signals and may extend to adjacent layers through via connections to provide three-dimensional shielding coverage.

Via placement and design within the PCB may be optimized to minimize electromagnetic interference and maintain signal integrity. High-speed digital signals may utilize via-in-pad techniques or closely positioned vias to minimize inductance and maintain controlled impedance transitions between layers. Ground vias may be positioned adjacent to signal vias to provide low impedance return paths and minimize electromagnetic radiation from via structures.

The PCB design may implement differential signaling techniques for critical high-speed data paths to improve noise immunity and reduce electromagnetic emissions. Differential pairs may be routed with matched trace lengths and controlled spacing to maintain consistent differential impedance. The differential traces may be positioned over continuous ground planes to provide stable reference potentials and minimize crosstalk between adjacent signal pairs.

Thermal management considerations within the PCB design may include thermal vias and copper pour areas to conduct heat away from high-power components. The LED Driver 52 and power regulation circuits may be positioned on areas of the PCB with enhanced thermal conductivity through increased copper thickness and thermal via arrays. The thermal design may prevent component overheating that could degrade performance or introduce thermal noise into sensitive analog circuits.

The PCB may incorporate decoupling capacitor placement strategies to minimize power supply noise and provide local energy storage for high-current switching events. Decoupling capacitors may be positioned as close as possible to power pins of integrated circuits to minimize inductance in the power delivery path. Multiple capacitor values may be used in parallel to provide effective decoupling across a wide frequency range from low-frequency power variations to high-frequency switching noise.

Trace routing techniques within the PCB may minimize electromagnetic interference through careful consideration of trace geometry and layer assignment. High-speed digital traces may be routed with controlled trace widths and spacing to maintain characteristic impedance and minimize crosstalk. Critical analog signals may be routed on inner layers surrounded by ground planes to provide electromagnetic shielding and reduce susceptibility to external interference sources.

The PCB design may implement clock distribution networks with controlled impedance and matched trace lengths to minimize clock skew and jitter. Clock signals may be routed on dedicated layers with appropriate termination to prevent reflections and maintain signal quality. Clock distribution may utilize buffer circuits positioned strategically to minimize propagation delays and provide adequate drive strength for multiple clock loads.

In another embodiment, the system may further include a comprehensive image reconstruction and analysis framework configured to transform raw optical measurements into clinically meaningful tissue characterization maps. The reconstruction framework may implement a multi-stage processing pipeline that may convert detected electromagnetic radiation into quantitative optical property distributions across the scanned tissue volume.

The image reconstruction framework may include a biologically guided initialization module configured to establish baseline optical property values based on known tissue absorption characteristics. The initialization module may assign initial absorption coefficient values for each pixel location based on published near-infrared absorption spectra of target tissue constituents. The initialization process may utilize reference absorption profiles for deoxyhemoglobin, oxyhemoglobin, water, and fat to provide physiologically realistic starting parameters for subsequent optimization algorithms.

The reconstruction framework may further include a reflectance normalization module configured to compensate for systematic variations in the optical measurement system. The normalization module may adjust measured pixel intensities to account for light source output variations, ambient light interference, integration time differences, and angle of incidence effects. The normalization process may apply calibration factors derived from reference measurements to ensure consistent optical property calculations across different scanning conditions and device orientations.

The system may implement a pixel-wise optimization engine configured to model photon transport pathways between source-detector pairs within the tissue volume. The optimization engine may represent photon propagation paths as banana-shaped sensitivity regions that may account for the curved trajectories of multiply scattered photons in turbid biological media. The optimization algorithm may minimize the error between measured reflectance values and theoretical predictions based on diffusion theory models of light transport in tissue.

The pixel-wise optimization may calculate reduced scattering coefficient and internal absorbance parameters for each spatial location within the imaging field. The optimization process may iteratively adjust these parameters to achieve optimal agreement between experimental measurements and theoretical reflectance predictions. The optimization algorithm may employ least-squares minimization techniques to determine the optical property values that may best explain the observed reflectance data at each wavelength.

The reconstruction framework may include an inverse modeling module configured to calculate absorption coefficients from the optimized scattering parameters and measured reflectance data. The inverse modeling module may apply wavelength-specific logarithmic inversion based on the Beer-Lambert law to determine absorption coefficient distributions across the tissue volume. The inversion process may account for the wavelength-dependent absorption characteristics of individual tissue chromophores to generate constituent-specific absorption maps.

The inverse modeling calculations may incorporate binning and smoothing operations to reduce measurement noise and enhance the biological relevance of the reconstructed absorption profiles. The binning process may average absorption values across adjacent pixel groups to improve signal-to-noise ratios while maintaining spatial resolution sufficient for clinical applications. The smoothing algorithms may apply spatial filtering techniques to suppress high-frequency noise artifacts that may arise from detector limitations or environmental interference.

The system may implement a multimodal alignment module configured to normalize absorption coefficient values across multiple light sources within the handheld probe. The alignment module may compensate for differences in light source output power, spectral characteristics, and geometric positioning that may introduce systematic variations between measurements from different emitters. The alignment process may utilize statistical scaling techniques or spatial registration algorithms to ensure consistent absorption coefficient calculations regardless of which light source provides the primary illumination.

The reconstruction framework may include a visualization engine configured to generate two-dimensional absorption coefficient heatmaps and three-dimensional volumetric reconstructions from the processed optical data. The visualization engine may render color-coded absorption maps that may highlight regions of elevated or reduced absorption relative to normal tissue baselines. The color mapping algorithms may apply physiologically relevant color scales that may facilitate clinical interpretation of tissue composition variations.

The visualization engine may implement interpolated three-dimensional reconstruction capabilities configured to generate volumetric tissue models from stacked cross-sectional absorption maps. The three-dimensional reconstruction process may utilize spline interpolation techniques to create smooth volumetric representations that may account for gaps between discrete measurement planes. The volumetric models may provide comprehensive visualization of tissue abnormalities and their spatial extent within the scanned tissue volume.

The system may include an area-under-the-curve integration module configured to quantify absorption coefficient distributions and convert them into geometric representations of tissue abnormalities. The integration module may calculate cumulative absorption values across spatial regions of interest and may transform these integrated values into circular cross-sectional profiles that may represent abnormal tissue volumes. The geometric transformation process may enable quantitative assessment of abnormality size, shape, and spatial distribution.

The reconstruction framework may implement a stacking engine configured to combine circular cross-sections from multiple imaging depths into three-dimensional abnormality models. The stacking process may align cross-sectional profiles according to their spatial coordinates and may generate volumetric representations of tissue abnormalities. The stacking algorithm may account for variations in cross-sectional size and position to create accurate three-dimensional models of abnormal tissue regions.

The system may include a longitudinal tracking module configured to monitor changes in tissue optical properties over extended time periods and quantify treatment response through comparative analysis of sequential imaging sessions. The longitudinal tracking module may compare absorption coefficient distributions, abnormality volumes, and spatial patterns across multiple imaging sessions to identify significant changes that may indicate treatment efficacy or disease progression.

The longitudinal tracking functionality may calculate residual cancer burden metrics based on changes in abnormal tissue volumes and absorption coefficient magnitudes following therapeutic interventions. The residual burden calculations may integrate absorption coefficient changes across three-dimensional tissue volumes and may provide quantitative measures of treatment response that may complement conventional imaging assessments. The tracking algorithms may account for patient positioning variations and probe placement differences between imaging sessions to ensure accurate temporal comparisons.

The system may implement synthetic data generation capabilities configured to enhance the accuracy and robustness of the optical property reconstruction algorithms through machine learning model training. The synthetic data generation module may convert clinical volumetric imaging data from modalities such as computed tomography, magnetic resonance imaging, or ultrasound into voxelated optical phantoms with assigned absorption and scattering coefficient distributions.

The synthetic data generation process may map voxel intensities from clinical images to wavelength-specific optical properties using predefined transfer functions based on known tissue characteristics. The mapping algorithms may assign absorption and reduced scattering coefficients to each voxel location based on the tissue type and density information contained in the source medical images. The resulting optical phantoms may serve as realistic tissue models for photon transport simulations and algorithm validation studies.

The system may utilize Monte Carlo photon transport simulations to generate synthetic reflectance data corresponding to the handheld probe geometry and measurement configuration. The Monte Carlo simulations may model photon propagation through the voxelated optical phantoms and may calculate reflectance profiles at detector locations corresponding to the actual device configuration. The simulation process may incorporate variance reduction techniques to accelerate convergence and may generate large datasets of labeled training samples for machine learning applications.

The synthetic data generation framework may implement stochastic variation algorithms configured to augment training datasets by introducing physiologically realistic variations in chromophore concentrations and optical properties. The augmentation process may randomly perturb absorption and scattering coefficients within physiological bounds to increase the diversity of training data and improve model generalizability. The stochastic variations may account for inter-patient variability and measurement uncertainties to create robust training datasets.

The system may deploy trained machine learning models on the onboard processor to provide real-time corrections and enhancements to the optical property reconstruction algorithms. The deployed models may utilize convolutional neural network architectures configured to jointly optimize absorption and scattering coefficient estimation across multiple wavelengths simultaneously. The neural network processing may operate in parallel with the physics-based reconstruction algorithms to provide improved accuracy and reduced computational requirements.

The machine learning integration may include model validation capabilities configured to verify algorithm performance using tissue-mimicking phantoms with known optical properties. The validation process may compare reconstructed optical property maps with ground truth phantom characteristics to quantify reconstruction accuracy and identify potential systematic errors. The validation framework may ensure that trained models maintain acceptable performance levels across diverse tissue types and measurement conditions before clinical deployment.

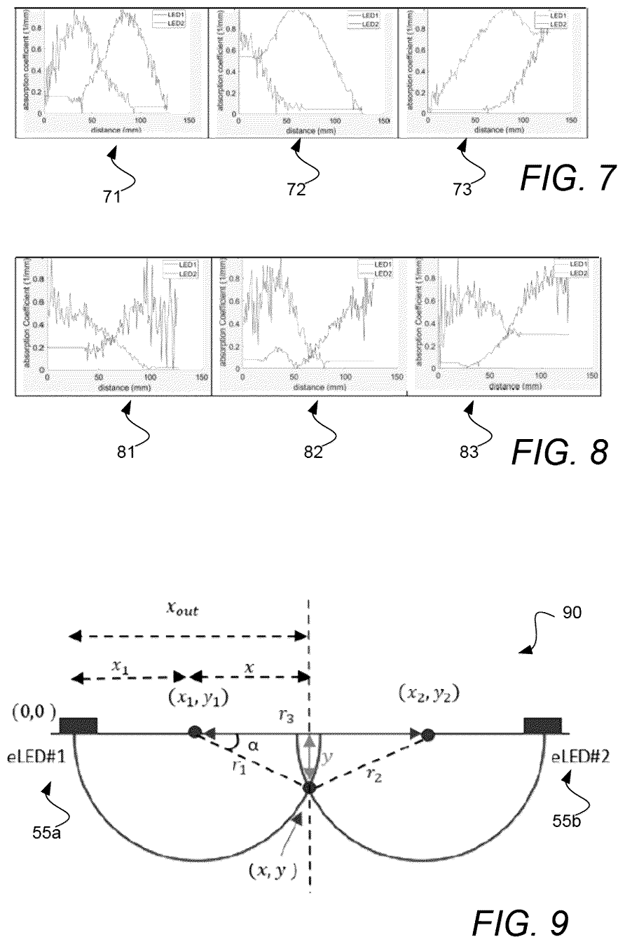

Turning attention to the methods and processes of the improved handheld probe, FIG. 7-17 are discussed next. One method particularly useful using the improved handheld probe 10 is breast cancer detection. Breast cancer detection and diagnosis may be crucial for effective treatment and management. While effective, current imaging techniques may face limitations in real-time functionality, accuracy, and the ability to provide comprehensive information on tissue abnormalities. There exists a need for an advanced imaging system that can deliver real-time, accurate, and detailed imaging of breast tissues to facilitate better diagnosis and treatment planning.

The optical properties of breast tissue, including the absorption coefficient (μa), may be critical in breast cancer diagnosis. The absorption coefficient may quantify the amount of light absorbed by tissue at a given depth, indicating the light absorption rather than scattering as it passes through the breast tissue. In diffuse reflectance theory, a diffusion coefficient (D) may be calculated using the reduced scattering coefficient (μ's), absorption coefficient (μa), and the anisotropy factor (g): D=μ's +μa/3, D=μ's/3(1−g). Further, in breast tissue, Depth (d) may refer to how deeply light can penetrate the tissue. The reduced scattering coefficient (μ's) may be expressed as: μ's=μs(1−g), μs=μ's/(1−g).

Using these expressions, the depth (d) may be calculated: d=1/√(3*μa(μa+μs(1−g))). Using the following μs+μa=μ's/(1−g)+μa=3μ's/(1−g)+3μa/3=(μ's/(1−g)+μa), we may get the depth (d), alternatively: d=√[(μa/μ's)D]/√(1−g).

A reflection coefficient (R) may measure the amount of light reflected from the tissue at a specific depth (d). It may be calculated using the refractive index (n) and impedance (z): n=(1−z)/(1+z), z=(1+n)/(1−n). The reflectance (R) for a semi-infinite homogeneous medium may be: R=((n−1)/(n+1))². Substituting these values, the reflection coefficient (R) may become: R=1/(4+d²+1).

An absorption coefficient (μa) may be calculated using: μa=−log(Rcollected*(4+d²+1))/((U0A/M0)*((U04+Rcollected)/3))+r, where Rcollected may represent the reflectance captured by the handheld probe 10, d may be the depth, and R may be the reflection coefficient derived from equations. By substituting the Rcollected, d, and R values into the equation, we may obtain μa, assuming a constant μs′.

Using data from a solid phantom with an abnormality at various positions (center, left, and right), we may calculate the absorption coefficients for LED1 and LED2. FIGS. 7-8 show these coefficients at 670 nm, highlighting an increasing trend towards the abnormality center and a subsequent decrease.

FIG. 7 shows absorption coefficients for a solid phantom with an abnormality (a) in the center, (b) offset to LED1 (left), and (c) offset to LED2 (right) according to an embodiment of the subject matter disclosed herein. The plot 71 may represent tissue scan data where an anomaly may be centered between the light sources. The plot 72 may represent tissue scan data where an anomaly may be positioned biased to left of the light sources. The plot 73 may represent tissue scan data where an anomaly may be positioned biased to the right of the light sources.

The absorption coefficient plots may demonstrate how the spatial positioning of tissue anomalies may affect the optical measurements captured by the handheld probe 10. The plot 71 may show absorption coefficient values when the anomaly may be equidistant from both light emitters 16. The centered positioning may result in balanced absorption characteristics across the measurement region. The plot 72 may show absorption coefficient values when the anomaly may be offset toward the left light emitter 16. The leftward positioning may result in asymmetric absorption patterns with higher values detected by the left-side optical components. The plot 73 may show absorption coefficient values when the anomaly may be offset toward the right light emitter. The rightward positioning may result in asymmetric absorption patterns with higher values detected by the right-side optical components.

The absorption coefficient measurements may be obtained at specific wavelengths corresponding to tissue constituent absorption characteristics. The wavelength selection may target chromophores such as deoxyhemoglobin, oxyhemoglobin, water, and fat within the tissue. The spatial variation in absorption coefficients may provide information about anomaly location and size relative to the probe positioning. The absorption coefficient data may be processed by the computing device 11 to generate cross-sectional images that may reveal the spatial distribution of tissue constituents and anomalies.

The plots 71-73 may demonstrate the capability of the handheld probe 10 to detect and localize tissue anomalies based on their optical absorption properties. The spatial sensitivity of the measurement system may enable differentiation between anomalies positioned at different locations within the tissue volume. The absorption coefficient variations may be used by the image reconstruction algorithms to generate accurate representations of tissue structure and composition.

FIG. 8 shows absorption coefficients for three different example patients corresponding to the examples of FIG. 7 according to an embodiment of the subject matter disclosed herein. These coefficients may inform the imaging algorithm used to map optical images, with further details provided in the next section. The absorption coefficient plots may be generated using the handheld probe 10 and computing device 11 described herein. Plot 81 may represent tissue scan data for a first patient. The first patient tissue scan may exhibit absorption coefficient characteristics that correspond to tissue abnormalities located in specific regions of the scanned tissue. Plot 82 may represent tissue scan data for a second patient. The second patient tissue scan may demonstrate absorption coefficient patterns that indicate tissue composition variations across the scanned area. Plot 83 may represent tissue scan data for a third patient. The third patient tissue scan may show absorption coefficient distributions that reveal tissue structural differences within the examined region.

The absorption coefficient plots 81, 82, and 83 may be obtained through the multi-wavelength illumination process described in connection with the handheld probe 10. Each plot may correspond to specific wavelength measurements captured by the light sensor 18 during tissue scanning procedures. The system may analyze the detected electromagnetic radiation to calculate absorption coefficients at various tissue depths and locations. The resulting absorption coefficient data may be processed to generate the visual representations shown in plots 81, 82, and 83.

FIG. 9 shows a diagrammatic representation 90 of algorithmic calculations for signals from two light emitters 16 labeled eLED #1 55a and eLED #2 55b at positions (0,0) and (x2, y2) according to an embodiment of the subject matter disclosed herein. FIG. 9 may illustrate the geometric configuration and algorithmic approach for determining light path intersections in the imaging system. The diagram may show two light emitter 16 sources positioned at coordinates (0,0) and (x2, y2), representing the spatial arrangement of the multi-wavelength light emitters 16 within the handheld probe 10. The eLED #1 55a may be positioned at the origin (x1, y1) or (0,0), while the eLED #2 55b may be located at coordinates (x2, y2) or (r3, 0). The distance r3 may represent the separation between the centers of the two semicircular light emission patterns generated by each light emitter 16.

The imaging algorithm may model the light propagation paths as semicircular regions emanating from each eLED source 55a and 55b. The radius r1 may correspond to the light path distance from eLED #1 55a to the intersection point, while radius r2 may represent the light path distance from eLED #2 55b to the same intersection point. The intersection point may be denoted as (x, y) relative to the coordinate system centered at the left eLED, or alternatively as (xout, yout) relative to the coordinate system located at LED1. This intersection point may represent the location within the tissue where light paths from both eLED sources 16 converge and where absorption coefficient calculations may be performed.

The algorithm may calculate the angle α (alpha) between the x-axis and the radius r1 using the law of cosines. The angle α may be determined from the relationship between the three distances r1, r2, and r3, where r1 and r2 may represent the radii of the semicircular light paths and r3 may represent the distance between the eLED centers. The calculation of α may enable the determination of the exact coordinates of the intersection point through trigonometric relationships.

The parameters r11 and r22 may calculate the distance from each source to the intersection point for LED1 and LED2, respectively. The coordinates x11 and x22 may represent the x-coordinate of the intersection point for LED1 and LED2, respectively, while y11 and y22 may initially be set to zero since the y-coordinate of the intersection point for both LEDs may be zero in the initial calculation. These coordinate values may be subsequently modified in step 3 of the algorithm based on the value of the depth parameter Δ.

The imaging algorithm may iterate through parameter values ranging from 0 to 127 for both LED1 and LED2, corresponding to the 128 measurement points derived from averaging 16 contiguous pixels from the 4096-pixel linear sensor 18. The source and detector separations may range from 15 mm to 43.4480 mm, with an effective imaging dimension of 28.672 mm. This systematic iteration may enable the algorithm to calculate absorption coefficients at multiple spatial locations within the tissue volume, providing the data necessary for generating cross-sectional images of tissue composition and structure.

FIG. 10 shows another diagrammatic representation 100 of algorithmic calculations for signals from two eLEDs labeled as eLED #1 55a and eLED #2 55b on each side of a central axis according to an embodiment of the subject matter disclosed herein. The diagram 100 may illustrate the geometric configuration and algorithmic approach for determining light path intersections when the light sources may be positioned symmetrically about a central detector axis. The eLED #1 55a may be positioned at coordinates (x1, y1), while the eLED #2 55b may be located at coordinates (x2, y2). The distance between the two light emitters may be represented by the separation along the horizontal axis.

The imaging algorithm may model the light propagation paths as curved regions emanating from each eLED source 55a and 55b. The radius r1 may correspond to the light path distance from eLED #1 55a to an intersection point, while radius r2 may represent the light path distance from eLED #2 55b to the same intersection point. The intersection point may be denoted as (x, y) and may represent the location within the tissue where light paths from both eLED sources converge. The convergence point may be where absorption coefficient calculations may be performed for tissue characterization.

The algorithm may calculate the angle α (alpha) between the central axis and the radius r1 using trigonometric relationships. The angle α may be determined from the geometric relationship between the light path distances and the source separation. The calculation of a may enable the determination of the exact coordinates of the intersection point through coordinate transformation equations. The intersection coordinates may be expressed as xnet=x1+x2 cos(α), where xnet may represent the net x-coordinate position of the convergence point.

The coordinates (xnet, ynet) may represent the final intersection point coordinates after geometric transformation. The ynet coordinate may be calculated using the sine component of the angle α and the corresponding radius values. These coordinate values may be used in subsequent processing steps to determine tissue optical properties at specific spatial locations within the scanned volume.

The imaging algorithm may iterate through multiple parameter combinations to calculate absorption coefficients at various spatial locations within the tissue volume. The systematic iteration may enable the algorithm to generate comprehensive optical property maps across the entire imaging field. The resulting data may provide the spatial information necessary for generating cross-sectional images of tissue composition and structure. The geometric relationships shown in FIG. 10 may form the mathematical foundation for the real-time image reconstruction algorithms implemented in the handheld probe system.

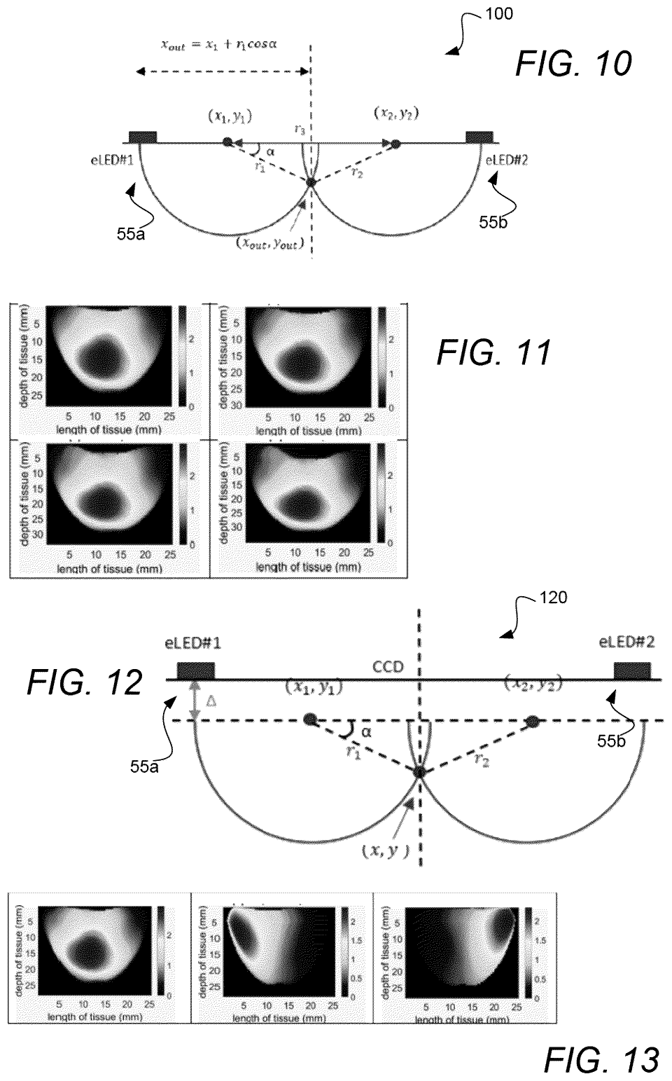

FIG. 11 shows four exemplary heat map images displaying data on the length and depth of tissue corresponding to the approach of FIG. 10 according to an embodiment of the subject matter disclosed herein. The four heat map images in FIG. 11 may demonstrate the imaging capabilities of the handheld probe system when applied to tissue characterization using the algorithmic approach described in FIG. 10. Each heat map may represent a cross-sectional view of tissue composition data obtained through multi-wavelength optical measurements. The heat maps may display spatial variations in tissue optical properties across both length and depth dimensions.

The first heat map may show tissue optical characteristics under baseline conditions. The color gradient may represent absorption coefficient variations, with red regions indicating areas of higher absorption and blue regions indicating areas of lower absorption. The spatial distribution may correspond to the natural heterogeneity of tissue constituents such as deoxyhemoglobin, oxyhemoglobin, water, and fat.

The second heat map may demonstrate the system's capability to detect and localize tissue anomalies. The central red region may indicate an area of increased optical absorption that may correspond to a tissue abnormality or lesion. The surrounding color transitions may represent the gradual change in optical properties as distance from the anomaly increases.

The third heat map may illustrate the system's sensitivity to variations in anomaly positioning. The optical absorption pattern may show asymmetric characteristics that may correspond to off-center positioning of tissue abnormalities. The color distribution may provide information about the spatial extent and optical characteristics of the detected anomaly.

The fourth heat map may represent tissue optical properties under different measurement conditions or at different wavelengths. The color pattern may demonstrate how multi-wavelength analysis may enhance the characterization of tissue composition and structure. The spatial resolution may enable differentiation between different tissue types and identification of regions of interest.

The length and depth axes may correspond to the physical dimensions of the tissue volume interrogated by the handheld probe. The length dimension may represent the spatial extent along the probe's scanning axis, while the depth dimension may indicate the penetration depth of the near-infrared radiation into the tissue. The color scale may provide quantitative information about the magnitude of optical property variations across the imaged tissue volume.

Next, one may evaluate this model by analyzing the data collected from the solid phantom, which may contain an abnormality located at the center. One may use an equation to generate the corresponding absorption coefficients. Then, the imaging algorithm may map these absorption values to the optical colored images. FIG. 11 may show regions with higher darkness or brown coloration, which may display the abnormality. The abnormality may be shown as a complete circular region with a diameter of about 5 mm. However, the depth estimation may remain approximately 22 mm, which may align with our initial expectations.

Depth Variation Correction: As described and shown in FIGS. 10 and 11, the algorithm's accuracy in estimating depth may have been compromised due to the neglect of changing light penetration depth within the tissue. To overcome this issue, one may incorporate the distance (Δ) calculation from the source point to a non-source point, as depicted in FIG. 12. The non-source point may be positioned at a distance of Δ.

FIG. 12 shows a diagrammatic representation 120 of algorithmic calculations for signals from two eLEDs labeled as eLED #1 and eLED #2 above a central axis according to an embodiment of the subject matter disclosed herein. The diagram 120 may illustrate an alternative geometric configuration where both light emitters 16 may be positioned on an offset side of the light sensor axis rather than in the axis. The eLED #1 55a may be positioned at coordinates (x1, y1), while the eLED #2 55b may be located at coordinates (x2, y2). Both light emitters may be positioned above the central detector axis to provide enhanced illumination coverage for specific tissue imaging applications.

The imaging algorithm may model the light propagation paths as curved regions emanating from each eLED source 55a and 55b positioned offset from the light sensor array. The radius r1 may correspond to the light path distance from eLED #1 55a to an intersection point within the tissue, while radius r2 may represent the light path distance from eLED #2 55b to the same intersection point. The intersection point may be denoted as (x, y) and may represent the location within the tissue where light paths from both eLED sources converge for absorption coefficient calculations.

The algorithm may calculate angles τ1 and τ2 between the vertical reference axis and the respective radii r1 and r2 using trigonometric relationships. The angles τ1 and τ2 may be determined from the geometric relationship between the light path distances and the source positions relative to the central detector axis. The calculation of these angles may enable the determination of the exact coordinates of the intersection point through coordinate transformation equations that account for the elevated positioning of both light sources.

The coordinates (x, y) may represent the intersection point coordinates after geometric transformation based on the angles τ1 and τ2 and the corresponding radius values. The intersection coordinates may be calculated using trigonometric functions that incorporate both angle measurements to determine the precise location where light paths from the elevated sources converge within the tissue volume. These coordinate values may be used in subsequent processing steps to determine tissue optical properties at specific spatial locations within the scanned volume.

The geometric configuration shown in FIG. 12 may provide advantages for certain tissue imaging applications where enhanced surface illumination may be beneficial. The elevated positioning of both light emitters may enable improved light penetration characteristics for tissues with specific optical properties or geometric constraints. The imaging algorithm may iterate through multiple parameter combinations to calculate absorption coefficients at various spatial locations within the tissue volume using the dual-elevated source configuration.

The systematic iteration may enable the algorithm to generate comprehensive optical property maps across the entire imaging field while accounting for the unique light path geometries created by the elevated source positioning. The resulting data may provide the spatial information necessary for generating cross-sectional images of tissue composition and structure. The geometric relationships shown in FIG. 12 may form an alternative mathematical foundation for the real-time image reconstruction algorithms implemented in the handheld probe system when dual-elevated source configurations may be advantageous for specific clinical applications.

FIG. 13 shows three exemplary heat map images displaying data on length and depth of tissue corresponding to the approach of FIG. 12 according to an embodiment of the subject matter disclosed herein. As before, FIG. 11 demonstrates the application of a solid phantom with an abnormality placed at the center, validating the new method's effectiveness. The obtained abnormality images may be shown, indicating the results obtained using different values of Δ ranges from −3 to 0. The best result, achieved with Δ=−3, may resolve the banana-shaped issue and accurately illustrate the abnormality's depth (15 mm) and length (4.5 mm). In FIG. 13, the reconstructed images may depict the solid phantom under different conditions: (a) with solution in the center, (b) with solution and offset to LED1 (left), and (c) with solution and offset to LED2 (right), where Δ=−3 at 670 nm.

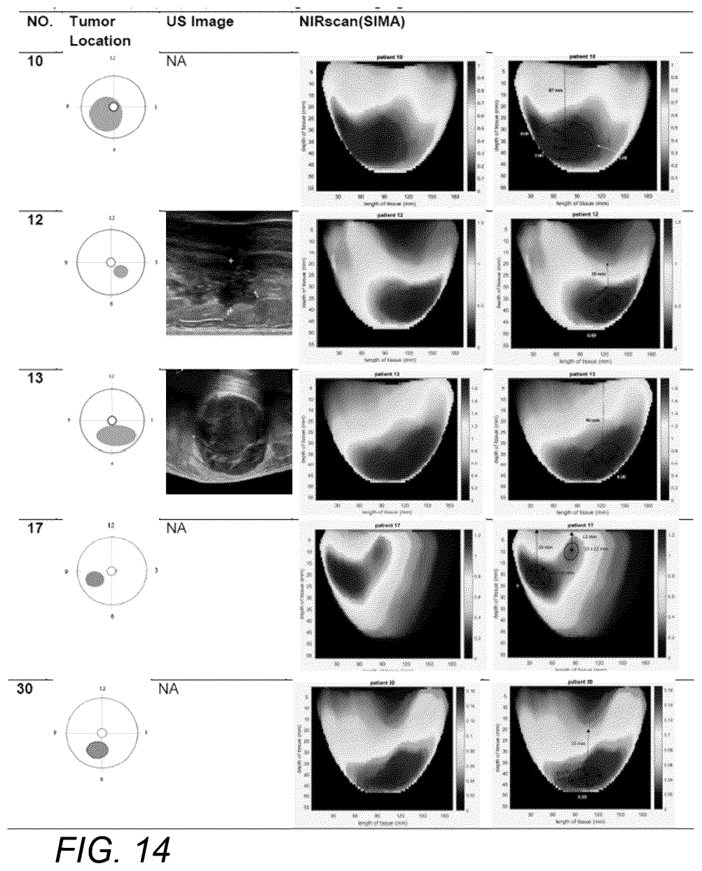

FIGS. 14-17 represent the results of the system's imaging methods applied to real breast cancer patient data. In contrast to the phantom experiments with a small abnormality, the patients in this study may have had tumors several centimeters in diameter, which may have exceeded the length of the light sensor 18. However, the probe 10 may have captured each slide representing the entire tumor by scaling down its size. Thus, the scaling of the x and y axes may be adjusted to encompass the complete tumor size.

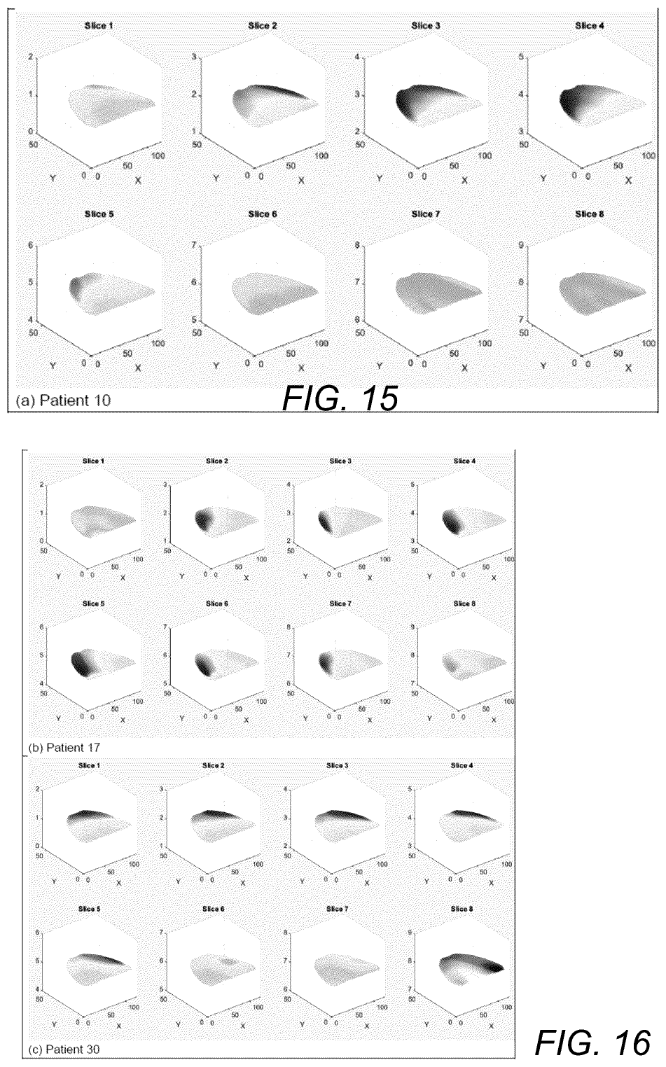

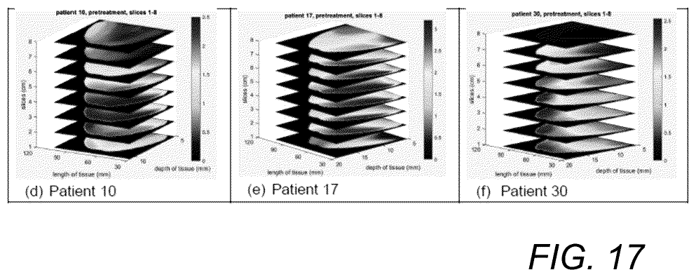

FIG. 14 shows exemplary tumor location diagrams having cross-sectional views with tumors marked, ultrasound images displaying portions of human tissue, and false-color images representing probe data with varying color gradients indicating different data points according to an embodiment of the subject matter disclosed herein. FIG. 15 shows a grid of eight exemplary 3D color-coded plots labeled “Slices 1 to 8” showing variations in data across axes labeled X, Y, Z according to an embodiment of the subject matter disclosed herein. FIG. 16 shows a set of sixteen exemplary 3D color-coded plots divided into two groups according to an embodiment of the subject matter disclosed herein. FIG. 17 shows exemplary 3D graphs with layers of colorful contours, transitioning from blue to red, representing patient data according to an embodiment of the subject matter disclosed herein.

These figures may show ultrasound data for five patients, with ultrasound positions adjusted by 180 degrees to match probe positions. Tumor size may be measured in terms of length×width×depth (mm), along with the approximate tumor location and the corresponding images from the cancerous breasts. This representation may enable a comparison of the probe's accuracy in measuring tumor position.

For example, patient 10 may have had a large tumor (60×70×39 mm) from position 5 to 7 and a small tumor (5×5×5 mm) at the 8 o'clock position. The probe 10 has accurately captured both tumors, including their size and positions. Patient 12 may have had a 32×13×20 mm tumor and another smaller tumor measuring 5×3×3 mm at 6 o'clock position (as per the palpation report, the tumor may be located at 5 o'clock). The probe 10 has correctly identified and sized tumors, with the larger tumor at 5 o'clock and the smaller one at 6 o'clock.

For patient 13's, ultrasound may have indicated a tumor size of 50×51×41 mm at 4:30 o'clock, and the probe 10 has precisely located and sized the tumor. For patient 17, a tumor measuring 30×30×20 mm may have been detected at 9 o'clock (although ultrasound may have reported 8 o'clock), along with a smaller tumor of 15×22×13 mm around 12 o'clock (not reported in ultrasound). Patient 30's ultrasound-measured tumor size is 37×35×23 mm at 6:30, and the probe 10 imaging has accurately captured the tumor's size and position.

To showcase 3D slice results, data imaging of different patients are shown in FIG. 16. Each slice may represent a cross-section of the tissue in 3D images, with the z-axis indicating slice depths and darker slices signifying tumor cross-sections. FIG. 17 offers a comprehensive view of all slices, collectively revealing the entire tumor.

While the subject matter discussed herein is susceptible to various modifications and alternative constructions, certain illustrated embodiments thereof are shown in the drawings and have been described above in detail. Furthermore, those skilled in the art will understand that various aspects described in less than all of the embodiments may, nevertheless, be present in any embodiment. It should be understood, however, that there is no intention to limit the subject matter to the specific forms disclosed, but on the contrary, the intention is to cover all modifications, alternative constructions, and equivalents falling within the spirit and scope of the subject matter disclosed.

1. An optical spectroscopy system for noninvasive imaging of human tissue, comprising:
a multi-wavelength near-infrared emitter source including at least one multi-chip light source configured to sequentially emit light at multiple wavelengths;
a local controller configured to control a light emission sequence at two or more wavelengths;
a sensor aligned in reflectance geometry with the emitter sources and configured to detect diffusely reflected near infrared light from biological tissue;
a remote processor configured to reconstruct images of tissue optical properties in real time; and
a sequential NIR illumination from at least one source aligned with the sensor detector.

2. The optical spectroscopy system of claim 1, wherein the multi-wavelength near-infrared emitter source comprises wavelengths selected from 670 nm, 810 nm, and 950 nm corresponding to absorption characteristics of one or more constituents selected from deoxyhemoglobin, oxyhemoglobin, water, lipids, fat, bone, cartilage, synovial fluid, and membrane.

3. The optical spectroscopy system of claim 1, wherein the at least one multi-chip light source comprises an encapsulated LED package containing multiple LED units for each wavelength.

4. The optical spectroscopy system of claim 1, wherein the sensor comprises a CMOS linear image sensor having 4096 pixels with high sensitivity in the near-infrared region.

5. The optical spectroscopy system of claim 1, wherein the local controller comprises a microcontroller unit configured to control wavelength sequencing, timing, and light source intensity.

6. The optical spectroscopy system of claim 1, wherein the remote processor is configured to reconstruct images at frame rates up to 60 frames per second.

7. The optical spectroscopy system of claim 1, further comprising a rechargeable power source with integrated power management circuitry.

8. The optical spectroscopy system of claim 1, wherein the system is configured in a handheld form factor comprising a multilayer printed circuit board optimized for noise suppression and thermal management.

9. The optical spectroscopy system of claim 1, further comprising auxiliary sensors configured to detect contact force and device orientation relative to the tissue surface.

10. The optical spectroscopy system of claim 1, wherein the multi-wavelength near-infrared emitter source further comprises at least one first light source oriented opposite the sensor to enable transmission-mode imaging through tissue and at least one second light source oriented outward for reflectance-mode imaging.

11. An optical spectroscopy imaging system for tissue characterization, comprising:
a handheld probe housing a multi-wavelength near-infrared light source configured to emit sequential illumination at wavelengths corresponding to tissue chromophore absorption spectra;
a linear detector array aligned in reflectance geometry with the light source and configured to capture diffusely reflected photons at multiple source-detector separations ;
a processing unit configured to execute real-time reconstruction algorithms for generating two-dimensional optical property maps; and
a user interface configured to display tissue composition images and control imaging parameters.

12. The optical spectroscopy imaging system of claim 11, wherein the multi-wavelength near-infrared light source comprises at least three wavelengths selected to target specific tissue constituents.

13. The optical spectroscopy imaging system of claim 11, wherein the linear detector array comprises a CMOS sensor with pixels optimized for near-infrared sensitivity and configured to measure photon intensity variations across spatial positions.

14. The optical spectroscopy imaging system of claim 11, wherein the processing unit is configured to apply diffusion theory modeling to reconstruct absorption and scattering coefficients from measured reflectance data.

15. The optical spectroscopy imaging system of claim 11, wherein the system is configured to classify breast tissue density into categories based on spatial distribution of reduced scattering coefficients.

16. The optical spectroscopy imaging system of claim 11, further comprising a longitudinal tracking module configured to monitor tissue changes over time and quantify treatment response.

17. An optical spectroscopy system for noninvasive imaging of human tissue, comprising:
a handheld probe comprising:
at least one multi-wavelength near-infrared light source configured to emit electromagnetic radiation at a plurality of wavelengths corresponding to absorption characteristics of human tissue constituents;
a linear detector array aligned in reflectance geometry with the at least one multi-wavelength near-infrared light source and configured to detect diffusely reflected electromagnetic radiation from the human tissue; and
a microcontroller unit configured to control sequential emission of the electromagnetic radiation at the plurality of wavelengths;
a processor coupled to the handheld probe and configured to:
receive detected electromagnetic radiation data from the linear detector array;
calculate absorption coefficients for each of the plurality of wavelengths;
reconstruct images of optical properties of the human tissue based on the calculated absorption coefficients; and
generate maps of tissue constituent concentrations at frame rates up to 60 frames per second; and
a multilayer printed circuit board configured to minimize electrical noise and optimize thermal management for the handheld probe.

18. A method for noninvasive tissue imaging using optical spectroscopy, comprising:
positioning a handheld probe containing a multi-wavelength near-infrared light source against tissue;
sequentially emitting light at multiple wavelengths corresponding to tissue chromophore absorption characteristics;
detecting diffusely reflected light using a sensor array aligned in reflectance geometry with the light source;
processing detected signals to calculate tissue optical properties including absorption and scattering coefficients; and
reconstructing images displaying spatial distribution of tissue constituents.

19. The method of claim 18, wherein the sequentially emitting comprises emitting light at wavelengths selected from 670 nm, 810 nm, and 950 nm to target deoxyhemoglobin, oxyhemoglobin, water, and fat absorption features.

20. A system for reconstructing spatial absorption and scattering maps of biological tissue using near-infrared diffuse reflectance data, the system comprising:
an image acquisition module configured to collect reflectance data from at least two wavelengths emitted from one or more multi-wavelength light sources and a detector array;
a biologically guided initialization module configured to assign initial spatial absorption values based on known tissue absorption profiles;
a reflectance normalization module that adjusts pixel intensities for light source variability, ambient light, integration time, and angle of incidence;
a pixel-wise optimization engine that models photon paths between source-detector pairs as regions of reduced scattering coefficient and internal absorbance parameter by minimizing an error between measured and theoretical reflectance;
a modeling engine computing total attenuation per pixel across wavelengths, followed by an inverse modeling module calculating spatial absorption using wavelength-specific logarithmic inversion based on the Beer-Lambert law;
a multimodal alignment module for normalizing spatial absorption values across multiple sources using statistical scaling or spatial registration techniques;
a visualization engine generating absorption heatmaps and rendering interpolated volumetric reconstructions from stacked cross-sections;
a longitudinal tracking module comparing abnormality volumes and spatial absorption distributions across time intervals to monitor treatment response;
a synthetic data generation module to update or fine-tune the pixel-wise optimization engine.

21. The system of claim 20, wherein the optimization engine minimizes mean squared error between theoretical and measured reflectance.

22. The system of claim 21 wherein the optimization engine further causes estimating chromophore concentrations using matrix-form Beer-Lambert law and least-squares fitting.

23. The system of claim 21, wherein the optimization engine is further configured to:
categorize breast tissue density into a first type, a second type, a third type, and a fourth type based upon one or more determined scattering coefficients, wherein:
the first type corresponds to predominantly fatty tissue with low scattering coefficient values;
the second type corresponds to scattered fibroglandular densities with moderate scattering coefficient values;
the third type corresponds to heterogeneously dense tissue with high scattering coefficient values; and
the fourth type corresponds to extremely dense tissue with uniformly elevated scattering coefficient values across a region of interest;
and wherein said classification supports individualized screening protocols and risk assessment in breast imaging.

24. The system of claim 20, wherein the normalization module corrects for light source output, integration time, ambient light, and incidence angle.

25. The system of claim 20, wherein the inverse modeling module applies binning and smoothing before computing biologically representative absorption curves.

26. The system of claim 20, wherein the alignment module uses statistical scaling to correct for asynchronous illumination.

27. The system of claim 20, wherein the visualization engine overlays source-detector paths on reconstructed images.
